(19) United States
(12) Patent Application Publication
(10) Pub. No.: US 2023/0061973 A1
(43) Pub. Date: Mar. 2, 2023

(54) TAT PEPTIDE BINDING PROTEINS AND USES THEREOF

(71) Applicants: Larimar Therapeutics, Inc., Bala Cynwyd, PA (US); The United States of America, as Represented by the Secretary, Dept. of Health and Human Services, Bethesda, MD (US)

(72) Inventors: Xin XU, Potomac, MD (US); Erik Johan Wagner, Sterling, VA (US); Bonita Rup, Boston, MA (US)

(21) Appl. No.: 17/796,561

(22) PCT Filed: Feb. 5, 2021
(86) PCT No.: PCT/US2021/016959

§ 371 Date: Jul. 29, 2022

Related U.S. Application Data
(60) Provisional application No. 62/970,662, filed on Feb. 5, 2020.

(51) Int. Cl. C07K 16/10; C12N 15/63

(52) U.S. Cl. CPC C07K 16/1072; C12N 15/63; C07K 2317/565; C07K 2317/622; C07K 2317/31; C07K 2317/24

(57) ABSTRACT

The present invention encompasses TAT peptide binding proteins. Specifically, the invention relates to antibodies that specifically bind to a TAT protein transduction domain. An antibody of the invention can be a full-length antibody or an antigen-binding portion thereof. Methods of making and methods of using the antibodies of the invention for detection, quantification, purification and isolation of TAT protein transduction domain, e.g., a TAT fusion molecule comprising a TAT protein transduction domain, as well as methods of diagnosis and monitoring of HIV and AIDS are also provided herein.

### RELATED APPLICATIONS

This application claims priority to U.S. Provisional Patent Application No. 62/970,662, filed on Feb. 5, 2020, the entire contents of which are hereby incorporated herein by reference.

### SEQUENCE LISTING

The instant application contains a Sequence Listing which has been submitted electronically in ASCII format and is hereby incorporated by reference in its entirety. Said ASCII copy, created on Feb. 5, 2021, is named 130197-00220_SL.txt and is 27,400 bytes in size.

### BACKGROUND

The human immunodeficiency virus type 1 (HIV-1) TAT protein is a key regulatory protein in the HIV-1 replication cycle. Wild-type TAT gene of HIV-1 is required for production of viral RNA and viral replication. TAT interacts with cellular transcriptional factors and cytokines, such as tumor necrosis factor-alpha (TNF-alpha), and alters the expression of a variety of genes in HIV-1-infected and non-infected cells.

TAT has also been shown to be taken up and internalized by cells. Thus, fusion of a heterologous protein to TAT has been utilized as a means for cellular delivery of heterologous proteins in cell culture and living animals.

The presence of TAT specific cytotoxic T lymphocytes is correlated with strong resistance to HIV infection (Allen et al. Nature 2000 407(6802):386 390). TAT mediated pathogenic effects can also be neutralized by anti-TAT antibodies. Antibodies directed against conserved regions of TAT, such as the cysteine rich and the lysine rich domains, have been shown to be particularly effective in inhibiting HIV replication. In HIV-1-infected patients, a strong humoral immune response against HIV-1 TAT protein is inversely correlated to peripheral blood viral load (Re et al. J. Clin. Virol. 2001 21(1):81 9).

There remains a need in the art for anti-TAT antibodies that can be used to detect and quantify TAT peptide, e.g., TAT peptide used as part of a fusion molecule for cellular delivery of a heterologous cargo moiety. There also remains a need for anti-TAT antibodies to be used for therapeutic purposes in the treatment of HIV infection.

### SUMMARY OF THE INVENTION

In certain aspects, the present invention provides for anti-TAT binding proteins, e.g., antibodies, and antigen binding portions thereof, that bind to a TAT protein transduction domain.

In one aspect, the present invention provides a binding protein comprising an antigen binding domain, the antigen binding domain comprising a heavy chain CDR3 domain comprising the amino acid sequence of SEQ ID NO: 4, wherein the binding protein is capable of binding a TAT protein transduction domain.

In some embodiments, the antigen binding domain further comprises a heavy chain CDR2 domain comprising the amino acid sequence of SEQ ID NO: 3.

In some embodiments, the antigen binding domain further comprises a heavy chain CDR1 domain comprising the amino acid sequence of SEQ ID NO: 2.

In some embodiments, the antigen binding domain further comprises a light chain CDR3 domain comprising the amino acid sequence selected from the group consisting of SEQ ID NO: 8 and SEQ ID NO: 12.

In some embodiments, the antigen binding domain further comprises a light chain CDR2 domain comprising the amino acid sequence selected from the group consisting of SEQ ID NO: 7 and SEQ ID NO:11.

In some embodiments, the antigen binding domain further comprises a light chain CDR1 domain comprising the amino acid sequence selected from the group consisting of SEQ ID NO: 6 and SEQ ID NO: 10.

In another aspect, the present invention provides a binding a protein comprising an antigen binding domain, the antigen binding domain comprising: a heavy chain variable region comprising a CDR3 domain comprising the amino acid sequence set forth in SEQ ID NO: 4, a CDR2 domain comprising the amino acid sequence set forth in SEQ ID NO: 3, and a CDR1 domain comprising the amino acid sequence set forth in SEQ ID NO: 2; and a light chain variable region comprising a CDR3 domain comprising the amino acid sequence set forth in SEQ ID NO: 8, a CDR2 domain comprising the amino acid sequence set forth in SEQ ID NO: 7, and a CDR1 domain comprising the amino acid sequence set forth in SEQ ID NO: 6; or a light chain variable region comprising a CDR3 domain comprising the amino acid sequence set forth in SEQ ID NO: 12, a CDR2 domain comprising the amino acid sequence set forth in SEQ ID NO: 11, and a CDR1 domain comprising the amino acid sequence set forth in SEQ ID NO: 10, wherein the binding protein is capable of binding a TAT protein transduction domain.

In some embodiments, the antigen binding domain comprises a heavy chain variable region comprising the amino acid sequence set forth in SEQ ID NO: 1 or SEQ ID NO: 14.

In some embodiments, the antigen binding domain comprises a light chain variable region comprising the amino acid sequence set forth in SEQ ID NO: 5, a light chain variable region comprising the amino acid sequence set forth in SEQ ID NO: 9, or a light chain variable region comprising the amino acid sequence set forth in SEQ ID NO: 13.

In some embodiments, the antigen binding domain comprises a heavy chain variable region comprising the amino acid sequence set forth in SEQ ID NO: 1 and a light chain variable region comprising the amino acid sequence set forth in SEQ ID NO: 5.

In some embodiments, the antigen binding domain comprises a heavy chain variable region comprising the amino acid sequence set forth in SEQ ID NO: 1 and a light chain variable region comprising the amino acid sequence set forth in SEQ ID NO: 9.

In some embodiments, the antigen binding domain comprises a heavy chain variable region comprising the amino acid sequence set forth in SEQ ID NO: 1 and a light chain variable region comprising the amino acid sequence set forth in SEQ ID NO: 13.

In some embodiments, the antigen binding domain comprises a heavy chain variable region comprising the amino acid sequence set forth in SEQ ID NO: 14 and a light chain variable region comprising the amino acid sequence set forth in SEQ ID NO: 5.

In another aspect, the present invention provides a binding protein comprising an antigen binding domain, the antigen binding domain comprising a heavy chain CDR3 domain comprising the amino acid sequence of SEQ ID NO: 18, wherein the binding protein is capable of binding a TAT protein transduction domain.

In some embodiments, the antigen binding domain further comprises a heavy chain CDR2 domain comprising the amino acid sequence of SEQ ID NO: 17.

In some embodiments, the antigen binding domain further comprises a heavy chain CDR1 domain comprising the amino acid sequence of SEQ ID NO: 16.

In some embodiments, the antigen binding domain further comprises a light chain CDR3 domain comprising the amino acid sequence of SEQ ID NO: 22.

In some embodiments, the antigen binding domain further comprises a light chain CDR2 domain comprising the amino acid sequence of SEQ ID NO: 21.

In some embodiments, the antigen binding domain further comprises a light chain CDR1 domain comprising the amino acid sequence of SEQ ID NO: 20.

In another aspect, the present invention provides a binding protein comprising an antigen binding domain, the antigen binding domain comprising: a heavy chain variable region comprising a CDR3 domain comprising the amino acid sequence set forth in SEQ ID NO: 18, a CDR2 domain comprising the amino acid sequence set forth in SEQ ID NO: 17, and a CDR1 domain comprising the amino acid sequence set forth in SEQ ID NO: 16; and a light chain variable region comprising a CDR3 domain comprising the amino acid sequence set forth in SEQ ID NO: 22, a CDR2 domain comprising the amino acid sequence set forth in SEQ ID NO: 21, and a CDR1 domain comprising the amino acid sequence set forth in SEQ ID NO: 20, wherein the binding protein is capable of binding a TAT protein transduction domain.

In some embodiments, the antigen binding domain comprises a heavy chain variable region comprising the amino acid sequence set forth in SEQ ID NO: 15.

In some embodiments, the antigen binding domain comprises a light chain variable region comprising the amino acid sequence set forth in SEQ ID NO: 19.

In some embodiments, the antigen binding domain comprises a heavy chain variable region comprising the amino acid sequence set forth in SEQ ID NO: 15 and a light chain variable region comprising the amino acid sequence set forth in SEQ ID NO: 19.

In some embodiments, the TAT protein transduction domain comprises the amino acid sequence of SEQ ID NO: 23.

In some embodiments, the TAT protein transduction domain consists essentially of the amino acid sequence of SEQ ID NO: 23.

In some embodiments, the TAT protein transduction domain is covalently linked to a cargo moiety.

In some embodiments, the cargo moiety is a polypeptide.

In some embodiments, the cargo moiety is a frataxin polypeptide.

In some embodiments, the cargo moiety is an antibody.

In some embodiments, the cargo moiety is a nucleic acid. In some embodiments, the nucleic acid is an siRNA, shRNA, miRNA, phosphorothioate modified RNA, aptamer, phosphorodiamidate morpholino oligomer (PMO), or any combination thereof.

In some embodiments, the cargo moiety is a small molecule, a liposome enclosing protein, a radionuclide or radionuclide labeled compound, or any combination thereof.

In some embodiments, the binding protein is capable of binding to a TAT protein transduction domain that is covalently linked to a cargo moiety.

In some embodiments, the antigen binding domain binds to an epitope comprising the amino acid residues of SEQ ID NO: 23.

In some embodiments, a binding protein disclosed herein is an antibody.

In another aspect, the present invention provides an antibody construct comprising a binding protein of the invention, further comprising a linker polypeptide or an immunoglobulin constant domain.

In some embodiments, binding protein is selected from the group consisting of: an immunoglobulin molecule, a monoclonal antibody, a murine antibody, a chimeric antibody, a CDR-grafted antibody, a humanized antibody, a single domain antibody, a Fv, a disulfide linked Fv, a scFv, a diabody, a Fab, a Fab′, a F(ab′)2, a multispecific antibody, a dual specific antibody, and a bispecific antibody.

In some embodiments, the antibody construct comprises a binding protein which comprises a heavy chain immunoglobulin constant domain selected from the group consisting of: a IgM constant domain, a IgG₄ constant domain, a IgG₁ constant domain, a IgE constant domain, a IgG₂ constant domain, a IgG₃ constant domain and a IgA constant domain.

In some embodiments, the heavy chain immunoglobulin constant domain is not IgM.

In another aspect, the present invention provides an isolated nucleic acid encoding a binding protein amino acid sequence of the invention.

In another aspect, the present invention provides an isolated nucleic acid encoding an antibody construct amino acid sequence of the invention.

In another aspect, the present invention provides a vector comprising an isolated nucleic acid according to the invention.

In some embodiments, the vector is selected from the group consisting of pcDNA, pTT, pTT3, pEFBOS, pBV, pJV, pBJ, pGEX, VSV, pBR322, pCMV-HA, pEN, YAC, BAC, Bacteriophage Lamda, Phagemid, pCAS9, pCEN6, pYES1L, p3HPRT1, pFN2A, pBC, pTZ, pGEM, pGEMK, pEX, pSAR, pCEP, Cosmids, pBluescript, pKJK, pFloxin, pCP, pHR, pUC, and pMAL.

In another aspect, the present invention provides a host cell comprising a vector according to the invention.

In some embodiments, the host cell is a prokaryotic cell or a eukaryotic cell. In some embodiments, the prokaryotic host cell is E. coli. In some embodiments, the eukaryotic cell is selected from the group consisting of a protist cell, an insect cell, an animal cell, a plant cell and a fungal cell. In some embodiments, the animal cell is a mammalian cell or an avian cell. In some embodiments, the host cell is selected from the group consisting of a CHO cell, a COS cell, a yeast cell, an insect Sf9 cell, HEK-293 cell, ExpiCHO cell, Expi-293f cell, and E. coli cell. In some embodiments, the yeast cell is Saccharomyces cerevisiae.

In another aspect, the present invention provides a method of producing an antibody, or antigen binding portion thereof, comprising culturing the host cell in culture medium so that the isolated nucleic acid is expressed and the antibody is produced.

In another aspect, the present invention provides an antibody produced according to the methods of the invention.

In another aspect, the present invention provides a transgenic mouse comprising the host cell described herein, wherein the mouse expresses a polypeptide encoded by the nucleic acid, or an antigen binding portion thereof, that binds to a TAT protein transduction domain.

In another aspect, the present invention provides a hybridoma that produces the antibody construct described herein.

In some embodiments, a binding protein according to the invention is immobilized on a solid support. In some embodiments, the solid support is a plate, a bead, or a chromatography resin. In some embodiments, the bead or chromatography resin comprises protein A agarose or protein G agarose.

In some embodiments, the binding protein of the invention is conjugated to a detection molecule.

In some embodiments, the detection molecule is horseradish peroxidase, sulfotag, alkaline phosphatase, cresol violet, quantum dots, FITC, an infrared molecule, a radiolabel, or an EPR spin tracer label.

In another aspect, the present invention provides a method for detecting and/or quantifying the level of a TAT fusion molecule in a sample, comprising contacting the sample with a binding protein of the invention under conditions such that the binding protein binds to TAT protein transduction domain in the sample, to thereby detect and/or quantify the level of the TAT fusion molecule in the sample.

In some embodiments, the sample is a biological sample.

In some embodiments, the biological sample is a liquid sample or a tissue sample.

In some embodiments, the TAT fusion molecule comprises a TAT protein transduction domain covalently linked to a cargo moiety.

In some embodiments, the cargo moiety is a polypeptide.

In some embodiments, the cargo moiety is a frataxin polypeptide.

In some embodiments, the cargomoiety is an antibody.

In some embodiments, the cargo moiety is a nucleic acid. In some embodiments, the nucleic acid is an siRNA, shRNA, miRNA, phosphorothioate modified RNA, aptamer, phosphorodiamidate morpholino oligomer (PMO), or any combination thereof.

In some embodiments, the cargo moiety is a small molecule, a liposome enclosing protein, a radionuclide or radionuclide labeled compound, or any combination thereof.

In some embodiments, the stability of the TAT fusion molecule is assessed.

In another aspect, the present invention provides method of isolating and/or purifying a TAT fusion molecule present in a mixture, wherein the TAT fusion molecule comprises a TAT protein transduction domain covalently linked to a cargo moiety, comprising (a) contacting the mixture comprising the TAT fusion molecule with the immobilized binding protein of the invention under conditions such that the TAT fusion molecule binds to the immobilized binding protein; (b) eluting the TAT fusion molecule from the immobilized binding protein.

In another aspect, the present invention provides a kit for comprising at least one reagent specific for the detection of a level of a TAT protein transduction domain, wherein the detection reagent is a binding protein of the invention. In some embodiments, the TAT protein transduction domain is covalently linked to a cargo moiety. In some embodiments, the kit further comprises instructions for the detection, quantitation or characterization of the TAT protein transduction domain.

In another aspect, the present invention provides a method of inhibiting the translocation of a TAT fusion molecule across a cell membrane, comprising contacting the TAT fusion molecule with an antigen binding protein of the invention, thereby inhibiting translocation of the TAT fusion molecule across the cell membrane.

In another aspect, the present invention provides a method of inhibiting the activity of HIV-TAT protein in a subject, comprising administering to the subject an antigen binding protein of the invention, thereby inhibiting activity of the HIV-TAT protein in the subject.

### BRIEF DESCRIPTION OF THE DRAWINGS

FIG. 1 illustrates that polyclonal antibodies raised against the entire TAT-FXN fusion molecule recognize drug, but not in a TAT specific manner.

FIG. 2 illustrates that polyclonal antibodies raised against KLH-TAT do not recognize mature frataxin, but do recognize BSA-TAT, showing specificity to the TAT epitope.

FIG. 3 is a bar graph showing relative peak area generated by a frataxin-derived tryptic peptide after immunopurification of an exemplary TAT frataxin fusion molecule using an anti-TAT rabbit polyclonal antibody (AC1058) and commercially available anti-frataxin antibodies (ab124680, ab113691 and ab110328) and a commercially available anti-TAT antibody (ab63957).

FIG. 4A and FIG. 4B show the ability of nine of ten anti TAT antibodies of the invention to capture an exemplary TAT frataxin fusion molecule in a pharmacokinetic (PK) assay in human plasma.

FIG. 5A shows that nine of ten anti TAT antibodies of the invention bind to an exemplary TAT frataxin fusion molecule in human plasma. FIG. 5B depicts the results of an anti-drug antibody (ADA) assay in human plasma, showing that the polyclonal anti-TAT antibody can function as a positive control in an ADA assay.

### DETAILED DESCRIPTION OF THE INVENTION

This invention pertains to TAT peptide binding proteins, particularly anti-TAT peptide antibodies, or antigen-binding portions thereof, that bind to a TAT protein transduction domain, including TAT fusion molecules comprising a TAT protein transduction domain, and uses thereof. Various aspects of the invention relate to antibodies and antibody fragments, conjugates thereof and pharmaceutical compositions thereof, as well as nucleic acids, recombinant expression vectors and host cells for making such antibodies and fragments. In one aspect, the invention pertains to a binding protein comprising an antigen binding domain, wherein the binding protein is capable of binding to a TAT protein transduction domain that is covalently linked to a cargo moiety. In some embodiments, the cargo moiety is a polypeptide. In some embodiments, the cargo moiety is a frataxin polypeptide. In some embodiments, the cargo moiety is an antibody.

In some embodiments, the cargo moiety is a pharmacologically active compound, a small molecule, a liposome enclosing protein, a radionuclide or radionuclide labeled compound, a nucleic acid, e.g., an siRNA, shRNA, miRNA, phosphorothioate modified RNA, aptamer, a phosphorodiamidate morpholino oligomer (PMO), or any combination thereof. Methods of using the binding proteins, e.g., antibodies, of the invention to detect and/or quantify the level of a TAT peptide, e.g., a TAT protein transduction domain, or a TAT fusion molecule in a sample, and to isolate and/or purify a TAT peptide or a TAT fusion molecule present in a mixture, are also encompassed by the invention. In some embodiments, the TAT fusion molecule comprises a TAT protein transduction domain covalently linked to a cargo moiety. In some embodiments, the cargo moiety is a polypeptide, e.g., a frataxin polypeptide.

In some embodiments, the cargo moiety is a pharmacologically active compound, a small molecule, a liposome enclosing protein, a radionuclide or radionuclide labeled compound, a nucleic acid, e.g., an siRNA, shRNA, miRNA, phosphorothioate modified RNA, aptamer, a phosphorodiamidate morpholino oligomer (PMO), or any combination thereof. In some embodiments, the methods of the invention further comprise assessing the stability of a TAT fusion molecule.

Methods of using the binding proteins, e.g., antibodies, of the invention to inhibit the translocation of a TAT fusion molecule across a cell membrane, and inhibit the activity of an HIV-TAT protein in a subject by determining the presence of the TAT protein or of part of the TAT protein in a biological sample are also encompassed by the invention. Methods of using the binding proteins, e.g., antibodies, of the invention to diagnose HIV infection in a subject are also encompassed by the invention.

### I. Definitions

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. The term “such as” is used herein to mean, and is used interchangeably, with the phrase “such as but not limited to.”

The articles “a” and “an” are used herein to refer to one or to more than one (i.e. to at least one) of the grammatical object of the article. By way of example, “an element” means one element or more than one element.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. As used herein, “one or more” is understood as each value 1, 2, 3, 4, 5, 6, 7, 8, 9, 10, and any value greater than 10.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the present invention may be more readily understood, select terms are defined below.

The term “polypeptide” or “peptide” as used herein, refers to any polymeric chain of amino acids. The term “protein” is used interchangeably with the terms peptide and polypeptide and also refer to a polymeric chain of amino acids. The term “peptide” or “polypeptide” encompasses native or artificial proteins, protein fragments and polypeptide analogs of a protein sequence. A polypeptide may be monomeric or polymeric.

The term “isolated protein” or “isolated polypeptide” or “isolated peptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art. An example of an isolated polypeptide is an isolated antibody, or antigen-binding portion thereof.

The term “TAT peptide” or “Trans-Activator of Transcription peptide” or “TAT”, as used interchangeably herein, refers to a trans-activating regulatory protein, or portion thereof. The TAT peptide is encoded by the lentivirus HIV-1. TAT peptide is essential for efficient transcription of the viral genome (Green and Lowenstein, 1988 Cell 55(6):1179-88). The full-length protein includes between 86 and 101 amino acids, depending on the subtype. In some embodiments, the full length TAT protein has the amino acid sequence set forth in SEQ ID NO: 24, as set forth below.

```
                10 20 30 40
MEPVDPRLEP WKHPGSQPKT ACTNCYCKKC CFHCQVCFIT
                50 60 70 80
KALGISYGRK KRRQRRRAHQ NSQTHQASLS KQPTSQPRGD
PTGPKE
```

In vivo, TAT increases the level of transcription of the HIV dsDNA. Before TAT is present, a small number of RNA transcripts are made, allowing the TAT protein to be produced. TAT then binds to cellular factors and mediates their phosphorylation, resulting in increased transcription of all HIV genes, providing a positive feedback cycle. TAT also appears to play a more direct role in the HIV disease process. TAT protein is released by infected cells in culture, and is found in the blood of HIV-1 infected patients.

TAT protein is able to translocate through the plasma membrane of cells and reach the nucleus to transactivate the viral genome. The “TAT protein transduction domain,” has been identified as responsible for cell penetration (Vives, et al., J Biol Chem. 1997 Jun. 20; 272(25):16010-7, the contents of which are hereby incorporated by reference).

As used herein the term “TAT protein transduction domain,” “TAT-PTD”, or “TAT translocation peptide”, as used interchangeably herein, refers to a TAT protein domain comprising amino acids 47-57 (amino acids YGRKKRRQRRR, SEQ ID NO: 23) of the 86 amino acid TAT protein (Frankel, et al. Cell, vol. 55, issue 6, 1988; M. Green, et al. Cell, vol. 55, issue 6, 1988; Fawell, et al. PNAS, 91(2), 664-668, 1994; U.S. Pat. No. 6,348,185, the contents of which are hereby incorporated herein by reference). In one embodiment, a TAT peptide antibody of the invention specifically binds to the TAT protein transduction domain. In one embodiment, the TAT protein transduction domain comprises the amino acid sequence of SEQ ID NO: 23 (YGRKKRRQRRR). In some embodiments, a TAT peptide binding protein of the invention specifically binds to a TAT protein transduction domain comprising the amino acid sequence of SEQ ID NO: 23. In another embodiment, a TAT peptide binding protein of the invention specifically binds to a TAT protein transduction domain consisting essentially of the amino acid sequence of SEQ ID NO: 23. In another embodiment, a TAT peptide binding protein of the invention specifically binds to a TAT protein transduction domain, e.g., SEQ ID NO: 23, which is contained in a TAT fusion molecule. For example, in one embodiment, the TAT fusion molecule comprises a TAT-frataxin fusion molecule.

As used herein a “TAT activity” or “TAT peptide activity” includes, but is not limited to, modulating, e.g., increasing, transcription of the viral genome, increasing the level of transcription of the HIV dsDNA, modulation of phosphorylation of cellular factors, and translocation of TAT through plasma membranes.

As used herein, the term “TAT fusion molecule” or “TAT fusion protein” refers to a TAT peptide, e.g., a TAT protein transduction domain, fused to a cargo moiety. The term “cargo moiety”, as used herein, refers to any molecule that can be transported into a cell when fused (e.g., covalently linked), to a TAT peptide, e.g., a TAT protein transduction domain. Thus, the cargo moiety is distinct from a TAT peptide or any fragment thereof. In one embodiment, the cargo moiety is transported in a non-pore forming manner. In one embodiment, the cargo moiety is a polypeptide. In one embodiment, the TAT fusion molecule is a TAT-frataxin fusion molecule, and the cargo moiety is a frataxin polypeptide. An exemplary TAT-frataxin fusion molecule is described, e.g., in PCT/US2020/044069, the entire contents of which are hereby incorporated herein by reference. In another embodiment, the cargo moiety is a therapeutic protein selected from the group consisting of Abarelix, Abatacept, Abciximab, Adalimumab, Aflibercept, Agalsidase beta, Albiglutide, Aldesleukin, Alefacept, Alemtuzumab, Alemtuzumab, Alglucerase, Alglucosidase alfa, Alirocumab, Aliskiren, Alpha-1-proteinase inhibitor, Alteplase, Anakinra, Ancestim, Anistreplase, Anthrax immune globulin human, Antihemophilic Factor, Anti-inhibitor coagulant complex, Antithrombin Alfa, Antithrombin III human, Antithymocyte globulin, Anti-thymocyte Globulin (Equine), Anti-thymocyte Globulin (Rabbit), Aprotinin, Arcitumomab, Asfotase Alfa, Asparaginase, Asparaginase Erwinia Chrysanthemi, Atezolizumab, Autologous cultured chondrocytes, Basiliximab, Becaplermin, Belatacept, Belimumab, Beractant, Bevacizumab, Bivalirudin, Blinatumomab, Botulinum Toxin Type A, Botulinum Toxin Type B, Brentuximab Vedotin, Brodalumab, Buserelin, C1 Esterase Inhibitor (Human), C1 Esterase Inhibitor (Recombinant), Canakinumab, Canakinumab, Capromab, Certolizumab Pegol, Cetuximab, Choriogonadotropin alfa, Chorionic Gonadotropin (Human), Chorionic Gonadotropin (Recombinant), Coagulation factor IX, Coagulation factor VIIa, Coagulation factor X human, Coagulation Factor XIII A-Subunit (Recombinant), Collagenase, Conestat alfa, Corticotropin, Cosyntropin, Daclizumab, Daptomycin, Daratumumab, Darbepoetin alfa, Defibrotide, Denileukin diftitox, Denosumab, Desirudin, Digoxin Immune Fab (Ovine), Dinutuximab, Dornase alfa, Drotrecogin alfa, Dulaglutide, Eculizumab, Efalizumab, Efmoroctocog alfa, Elosulfase alfa, Elotuzumab, Enfuvirtide, Epoetin alfa, Epoetin zeta, Eptifibatide, Etanercept, Evolocumab, Exenatide, Factor IX Complex (Human), Fibrinogen Concentrate (Human), Fibrinolysin (Plasmin), Filgrastim, Filgrastim-sndz, Follitropin alpha, Follitropin beta, Galsulfase, Gastric intrinsic factor, Gemtuzumab ozogamicin, Glatiramer acetate, Glucagon recombinant, Glucarpidase, Golimumab, Gramicidin D, Hepatitis A Vaccine, Hepatitis B immune globulin, Human calcitonin, Human Clostridium tetani toxoid immune globulin, Human rabies virus immune globulin, Human Rho(D) immune globulin, Human Serum Albumin, Human Varicella-Zoster Immune Globulin, Hyaluronidase, Hyaluronidase (Human Recombinant), Ibritumomab, Ibritumomab tiuxetan, Idarucizumab, Idursulfase, Imiglucerase, Immune Globulin Human, Infliximab, Insulin Aspart, Insulin, Gemtuzumab ozogamicin, Glatiramer acetate, Glucagon recombinant, Glucarpidase, Golimumab, Gramicidin D, Hepatitis A Vaccine, Hepatitis B immune globulin, Human calcitonin, Human Clostridium tetani toxoid immune globulin, Human rabies virus immune globulin, Human Rho(D) immune globulin, Human Serum Albumin, Human Varicella-Zoster Immune Globulin, Hyaluronidase, Hyaluronidase (Human Recombinant), Ibritumomab, Ibritumomab tiuxetan, Idarucizumab, Idursulfase, Imiglucerase, Immune Globulin Human, Infliximab, Insulin aspart, Insulin Beef, Insulin Degludec, Insulin detemir, Insulin Glargine, Insulin glulisine, Insulin Lispro, Insulin (Pork), Insulin Regular, Insulin (porcine), Insulin-isophane, Interferon Alfa-2a, Recombinant, Interferon alfa-2b, Interferon alfacon-1, Interferon alfa-n1, Interferon alfa-n3, Interferon beta-1a, Interferon beta-1b, Interferon gamma-1b, Intravenous Immunoglobulin, Ipilimumab, Ixekizumab, Laronidase, Lenograstim, Lepirudin, Leuprolide, Liraglutide, Lucinactant, Lutropin alfa, Mecasermin, Menotropins, Mepolizumab, Methoxy polyethylene glycol-epoetin beta, Metreleptin, Muromonab, Natalizumab, Natural alpha interferon OR multiferon, Necitumumab, Nesiritide, Nivolumab, Obiltoxaximab, Obinutuzumab, Ocriplasmin, Ofatumumab, Omalizumab, Oprelvekin, OspA lipoprotein, Oxytocin, Palifermin, Palivizumab, Pancrelipase, Panitumumab, Pegademase bovine, Pegaptanib, Pegaspargase, Pegfilgrastim, Peginterferon alfa-2a, Peginterferon alfa-2b, Peginterferon beta-1a, Pegloticase, Pegvisomant, Pembrolizumab, Pertuzumab, Poractant alfa, Pramlintide, Preotact, Protamine sulfate, Protein S human, Prothrombin complex concentrate, Ragweed Pollen Extract, Ramucirumab, Ranibizumab, Rasburicase, Raxibacumab, Reteplase, Rilonacept, Rituximab, Romiplostim, Sacrosidase, Salmon Calcitonin, Sargramostim, Satumomab Pendetide, Sebelipase alfa, Secretin, Secukinumab, Sermorelin, Serum albumin, Serum albumin iodonated, Siltuximab, Simoctocog Alfa, Sipuleucel-T, Somatotropin Recombinant, Somatropin recombinant, Streptokinase, Sulodexide, Susoctocog alfa, Taliglucerase alfa, Teduglutide, Teicoplanin, Tenecteplase, Teriparatide, Tesamorelin, Thrombomodulin Alfa, Thymalfasin, Thyroglobulin, Thyrotropin Alfa, Tocilizumab, Tositumomab, Trastuzumab, Tuberculin Purified Protein Derivative, Turoctocog alfa, Urofollitropin, Urokinase, Ustekinumab, Vasopressin, Vedolizumab, Velaglucerase alfa, Thrombomodulin Alfa, Thymalfasin, Thyroglobulin, Thyrotropin Alfa, Tocilizumab, Tositumomab, Trastuzumab, Tuberculin Purified Protein Derivative, Turoctocog alfa, Urofollitropin, Urokinase, Ustekinumab, Vasopressin, Vedolizumab, Velaglucerase alfa. In another embodiment, the protein is selected from those included in Raghava, Gajendra P. S.; Usmani, Salman Sadullah; Bedi, Gursimran; Samuel, Jesse S.; Singh, Sandeep; Kalra, Sourav; et al. (2017): THPdb: Database of FDA Approved Peptide and Protein Therapeutics, the contents of which are hereby incorporated herein by reference.

In one embodiment, a polypeptide cargo moiety may be between about 100 kD and 200 nM in size. In another embodiment, the cargo moiety is a pharmacologically active compound, a small molecule, a liposome enclosing protein, a radionuclide or radionuclide labeled compound, a nucleic acid, e.g., an siRNA, shRNA, miRNA, phosphorothioate modified RNA, aptamer, a phosphorodiamidate morpholino oligomer (PMO), or any combination thereof. In another embodiment, the TAT fusion molecule is a tissue penetrant TAT fusion molecule.

As used herein, the term “TAT-frataxin fusion molecule” includes any fusion molecule comprising a TAT protein transduction domain, or a fragment thereof, and a frataxin peptide, or a fragment thereof. In one embodiment, fusion of the frataxin polypeptide to a TAT peptide (e.g., the TAT protein transduction domain), allows for translocation of the entire fusion protein through the cell membrane. Exemplary TAT-frataxin fusion molecules are described in U.S. Pat. No. 8,283,444, the contents of which is hereby incorporated herein by reference.

As used herein, “detecting”, “detection”, “determining”, and the like are understood that an assay performed for identification of a specific antigen in a sample, e.g., a TAT protein transduction domain, or a TAT fusion molecule, such as a TAT-frataxin fusion molecule. Detection can be by any means known in the art. For example, a TAT protein transduction domain, or a TAT fusion molecule, such as a TAT-frataxin fusion molecule can be detected by contacting a sample comprising the TAT protein transduction domain or TAT fusion molecule, with a binding protein of the invention under conditions such that the binding protein binds to the TAT protein transduction domain in the sample. In one embodiment, the binding protein is immobilized. In another embodiment, the TAT transduction domain or the TAT fusion molecule is eluted from the immobilized binding protein.

The terms “specific binding” or “specifically binding”, as used herein, in reference to the interaction of a binding protein, e.g., an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

In one embodiment, the phrase “specifically binds to TAT a TAT protein transduction domain” or “specific binding to a TAT protein transduction domain” as used herein, refers to the ability of an anti-TAT binding protein to interact with a TAT protein transduction domain with a dissociation constant (K_{D}) of about 2,000 nM or less, about 1,000 nM or less, about 500 nM or less, about 200 nM or less, about 100 nM or less, about 75 nM or less, about 50 nM or less, about 43 nM or less, about 25 nM or less, about 20 nM or less, about 19 nM or less, about 18 nM or less, about 17 nM or less, about 16 nM or less, about 15 nM or less, about 14 nM or less, about 13 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, or about 0.01 nM or less, or about 0.001 nM or less.

In another embodiment, the phrase “specifically binds to a TAT protein transduction domain” or “specific binding to a TAT protein transduction domain”, as used herein, refers to the ability of an anti-TAT binding protein to interact with a TAT protein transduction domain with a dissociation constant (K_{D}) of between about 1 pM (0.001 nM) to 2,000 nM, between about 500 pM (0.5 nM) to 1,000 nM, between about 500 pM (0.5 nM) to 500 nM, between about 1 nM) to 200 nM, between about 1 nM to 100 nM, between about 1 nM to 50 nM, between about 1 nM to 20 nM, or between about 1 nM to 5 nM. In one embodiment, K_{D} is determined by surface plasmon resonance.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In a preferred embodiment, the immunoglobulin molecules are IgG1.

The term “antigen-binding portion” of an antibody (or simply “antibody portion”), as used herein, refers to one or more fragments of an antibody that retain the ability to specifically bind to an antigen (e.g., a TAT protein transduction domain). It has been shown that the antigen binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding portion” of an antibody. In certain embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more the antigen binding portions disclosed herein linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated binding protein” or “isolated antibody”, as used herein, is intended to refer to a binding protein, e.g., antibody, that is substantially free of other binding proteins having different antigenic specificities (e.g., an isolated antibody that specifically binds a TAT protein transduction domain is substantially free of antibodies that specifically bind antigens other than a TAT protein transduction domain). Moreover, an isolated binding protein may be substantially free of other cellular material and/or chemicals.

The term “humanized antibody” refers to antibodies which comprise heavy and light chain variable region sequences from a nonhuman species (e.g., a mouse) but in which at least a portion of the VH and/or VL sequence has been altered to be more “human-like”, i.e., more similar to human germline variable sequences. In particular, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In other embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG1, IgG2, IgG3 and IgG4. The humanized antibody may comprise sequences from more than one class or isotype, and particular constant domains may be selected to optimize desired effector functions using techniques well-known in the art.

The terms “Kabat numbering,” “Kabat definitions,” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The term “multivalent antibody” is used herein to denote an antibody comprising two or more antigen binding sites. In certain embodiments, the multivalent antibody may be engineered to have the three or more antigen binding sites, and is generally not a naturally occurring antibody.

The term “multispecific antibody” refers to an antibody capable of binding two or more unrelated antigens.

The term “dual variable domain” or “DVD,” as used interchangeably herein, are antigen binding proteins that comprise two or more antigen binding sites and are tetravalent or multivalent binding proteins. Such DVDs may be monospecific, i.e., capable of binding one antigen or multispecific, i.e. capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. In one embodiment, the CDRs described herein are used in an anti-TAT DVD.

The term “activity” includes activities such as the binding specificity/affinity of a binding protein, e.g., an antibody, for an antigen, for example, an anti-TAT antibody that binds to a TAT protein transduction domain antigen. In one embodiment, an anti-TAT antibody activity includes, but it not limited to, binding to a TAT protein transduction domain in vitro; binding to a TAT protein transduction domain in vivo; and decreasing or inhibiting HIV infection.

The term “epitope” refers to a region of an antigen that is bound by a binding protein, e.g., an antibody or antibody portion. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In certain embodiments, an antibody is said to specifically bind an antigen when it preferentially recognizes its target antigen in a complex mixture of proteins and/or macromolecules.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “kₒₙ” or “kₐ”, as used herein, is intended to refer to the on rate constant for association of an antibody to the antigen to form the antibody/antigen complex.

The term “k_{off}” or “kₐ”, as used herein, is intended to refer to the off rate constant for dissociation of an antibody from the antibody/antigen complex.

The term “K_{D}”, as used herein, is intended to refer to the equilibrium dissociation constant of a particular antibody-antigen interaction. K_{D} is calculated by kₐ/k_{d}.

The term “competitive binding”, as used herein, refers to a situation in which a first antibody competes with a second antibody, for a binding site on a third molecule, e.g., an antigen. In one embodiment, competitive binding between two antibodies is determined using FACS analysis.

The term “competitive binding assay” is an assay used to determine whether two or more antibodies bind to the same epitope. In one embodiment, a competitive binding assay is a competition fluorescent activated cell sorting (FACS) assay which is used to determine whether two or more antibodies bind to the same epitope by determining whether the fluorescent signal of a labeled antibody is reduced due to the introduction of a non-labeled antibody, where competition for the same epitope will lower the level of fluorescence.

The term “labeled binding protein” as used herein, refers to a binding protein, e.g., an antibody, with a label incorporated that provides for the identification of the binding protein or the target moiety. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In ¹²⁵In, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; biotin, digoxigenin, predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “polynucleotide” as used herein refers to a polymeric form of two or more nucleotides, either ribonucleotides or deoxvnucleotides or a modified form of either type of nucleotide. The term includes single and double stranded forms of DNA but preferably is double-stranded DNA.

The term “isolated polynucleotide” as used herein shall mean a polynucleotide (e.g., of genomic, cDNA, or synthetic origin, or some combination thereof) that, by virtue of its origin, the “isolated polynucleotide”: is not associated with all or a portion of a polynucleotide with which the “isolated polynucleotide” is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Exemplary vectors include, for example, pcDNA, pTT, pTT3, pEFBOS, pBV, pJV, pBJ, pGEX, VSV, pBR322, pCMV-HA, pEN, YAC, BAC, Bacteriophage Lamda, Phagemid, pCAS9, pCEN6, pYES1L, p3HPRT1, pFN2A, pBC, pTZ, pGEM, pGEMK, pEX, pSAR, pCEP, Cosmids, pBluescript, pKJK, pFloxin, pCP, pHR, pUC, and pMAL. Additional vectors are included in International Publication No. WO2005108568, the contents of which are incorporated herein by reference.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences. Protein constructs of the present invention may be expressed, and purified using expression vectors and host cells known in the art, including expression cassettes, vectors, recombinant host cells and methods for the recombinant expression and proteolytic processing of recombinant polyproteins and pre-proteins from a single open reading frame (e.g., WO 2007/014162 incorporated herein by reference).

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. coli; mammalian cell lines CHO, HEK 293 and COS; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, serum, urine, synovial fluid, cells, organs, tissues, bone marrow, lymph nodes and spleen.

A “patient” or “subject” to be treated by the method of the invention can mean either a human or non-human animal, preferably a mammal. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient. It should be noted that clinical observations described herein were made with human subjects and, in at least some embodiments, the subjects are human.

The terms “disorders” and “diseases” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. Certain characteristic signs, symptoms, and related factors can be quantitated through a variety of methods to yield important diagnostic information. As used herein a “TAT associated disease” includes human immunodeficiency virus (HIV) infection or Acquired immunodeficiency syndrome (AIDS), and symptoms caused by or related to HIV infection or AIDS.

The terms “human immunodeficiency virus” or “HIV,” as used herein, refer generally to a retrovirus that is the causative agent for acquired immunodeficiency syndrome (AIDS), variants thereof (e.g., simian acquired immunodeficiency syndrome, SAIDS), and diseases, conditions, or opportunistic infections associated with AIDS or its variants, and includes HIV-Type 1 (HIV-1) and HIV-Type 2 (HIV-2) of any Glade or strain therein, related retroviruses (e.g., simian immunodeficiency virus (SIV)), and variants thereof (e.g., engineered retroviruses, e.g., chimeric HIV viruses, e.g., simian-human immunodeficiency viruses (SHIVs)). Previous names for HIV include human T-lymphotropic virus-Ill (HTLV-III), lymphadenopathy-associated virus (LAV), and AIDS-associated retrovirus (ARV).

As used herein, and as well understood in the art, “treatment” is an approach for obtaining beneficial or desired results, such as clinical results. Beneficial or desired results can include, but are not limited to, cure or eradication of disease, disorder, or condition (e.g., HIV infection); alleviation or amelioration of one or more symptoms or conditions (e.g., HIV infection); diminishment of extent of disease, disorder, or condition (e.g., HIV infection); stabilization (i.e., not worsening) of a state of disease, disorder, or condition (e.g., HIV infection); prevention of spread or transmission of disease, disorder, or condition (e.g., HIV infection); delay or slowing the progress of the disease, disorder, or condition (e.g., HIV infection); amelioration or palliation of the disease, disorder, or condition (e.g., HIV infection); and remission (whether partial or total), whether detectable or undetectable.

As used herein, by “curing” a subject (e.g., a human) infected with a retrovirus (e.g., human immunodeficiency virus (HIV) (e.g., HIV Type 1 or HIV Type 2)) is meant obtaining and maintaining virologic control in the absence of an antiretroviral therapy (ART) for a period of at least two months (e.g., 2 months, 2.5 months, 3 months, 4 months, 5 months, 6 months, 1 year, 1.5 years, 2 years, 3 years, 4 years, or 5 or more years).

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

As used herein, the term “obtaining” is understood herein as manufacturing, purchasing, or otherwise coming into possession of.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### II. Binding Proteins that Bind TAT Peptide

One aspect of the present invention provides isolated binding proteins, e.g., antibodies, or antigen-binding portions thereof, that bind to a TAT peptide, and specifically that bind to a TAT protein transduction domain. Anti-TAT peptide antibodies and methods of making the binding proteins, methods of producing the binding proteins are also described in detail herein below.

A. Anti-TAT Peptide Binding Proteins

The present invention features binding proteins, e.g., antibodies, comprising an antigen binding domain, said binding protein capable of binding a TAT peptide. In particular, said binding protein is capable of binding a TAT protein transduction domain. Collectively, the novel antibodies are referred to herein as “TAT peptide antibodies” or “anti-TAT antibodies.”

While the term “antibody” is used throughout, it should be noted that antibody portions (i.e., antigen-binding portions of an anti-TAT antibody) are also included in the disclosure and may be included in the embodiments (methods and compositions) described throughout. For example, an anti-TAT antibody portion may be conjugated to the drugs, as described herein. In certain embodiments, an anti-TAT antibody binding portion is a Fab, a Fab′, a F(ab′)2, a Fv, a disulfide linked Fv, an scFv, a single domain antibody, or a diabody.

A list of amino acid sequences of VH and VL regions, as well as CDRs, of preferred monoclonal antibodies of the invention are shown in Table 1 of Example 1.

In one aspect, the invention features a binding protein comprising an antigen binding domain, said binding protein capable of binding a TAT protein transduction domain, said antigen binding domain comprising a heavy chain variable region comprising an amino acid sequence selected from the group consisting of SEQ ID NOs: 1, 14, and 15; and a light chain variable region comprising an amino acid sequence selected from the group consisting of 5, 9, 13, and 19.

In one aspect, the invention features a binding protein comprising an antigen binding domain, said binding protein capable of binding a TAT protein transduction domain, said antigen binding domain comprising a heavy chain CDR set (CDR1, CDR2, and CDR3) selected from those set forth in Table 1; and an LC CDR set (CDR1, CDR2, and CDR3) selected from those set forth in Table 1.

In one aspect, the invention features a binding protein comprising an antigen binding domain, said binding protein capable of binding a TAT protein transduction domain, said antigen binding domain comprising a heavy chain CDR3 domain comprising the amino acid sequence selected from the group consisting of SEQ ID NO: 4 or SEQ ID NO: 18. In one embodiment, the binding protein further comprises a heavy chain CDR2 domain comprising the amino acid sequence selected from the group consisting of SEQ ID NO: 3 or SEQ ID NO: 17. In another embodiment, the binding protein further comprises a heavy chain CDR1 domain comprising the amino acid sequence selected from the group consisting of SEQ ID NO: 2 or SEQ ID NO: 16. In another embodiment, the binding protein further comprises a light chain CDR3 domain comprising the amino acid sequence selected from the group consisting of SEQ ID NO: 8, SEQ ID NO: 12, or SEQ ID NO: 22. In another embodiment, the binding protein further comprises a light chain CDR2 domain comprising the amino acid sequence selected from the group consisting of SEQ ID NO: 7, SEQ ID NO: 11, or SEQ ID NO: 21. In another embodiment, the binding protein further comprises a light chain CDR1 domain comprising the amino acid sequence selected from the group consisting of SEQ ID NO: 6, SEQ ID NO: 10, or SEQ ID NO: 20.

In another aspect, the invention features a binding protein comprising an antigen binding domain, said binding protein capable of binding a TAT protein transduction domain, said antigen binding domain comprising a heavy chain variable region comprising a CDR3 domain comprising the amino acid sequence set forth in SEQ ID NO: 4, a CDR2 domain comprising the amino acid sequence set forth in SEQ ID NO: 3, and a CDR1 domain comprising the amino acid sequence set forth in SEQ ID NO: 2 or a heavy chain variable region comprising a CDR3 domain comprising the amino acid sequence set forth in SEQ ID NO:18, a CDR2 domain comprising the amino acid sequence set forth in SEQ ID NO: 17, and a CDR1 domain comprising the amino acid sequence set forth in SEQ ID NO: 16 or; and a light chain variable region comprising a CDR3 domain comprising the amino acid sequence set forth in SEQ ID NO: 8, a CDR2 domain comprising the amino acid sequence set forth in SEQ ID NO: 7, and a CDR1 domain comprising the amino acid sequence set forth in SEQ ID NO: 6, or a light chain variable region comprising a CDR3 domain comprising the amino acid sequence set forth in SEQ ID NO: 12, a CDR2 domain comprising the amino acid sequence set forth in SEQ ID NO: 11, and a CDR1 domain comprising the amino acid sequence set forth in SEQ ID NO: 10, or a light chain variable region comprising a CDR3 domain comprising the amino acid sequence set forth in SEQ ID NO: 22, a CDR2 domain comprising the amino acid sequence set forth in SEQ ID NO: 21, and a CDR1 domain comprising the amino acid sequence set forth in SEQ ID NO: 20.

The present invention also features in other aspects, a binding protein comprising an antigen binding domain, said binding protein capable of binding a TAT protein transduction domain, said antigen binding domain comprising a heavy chain variable region comprising a CDR3 domain comprising the amino acid sequence of SEQ ID NO: 4, a CDR2 domain comprising the amino acid sequence of SEQ ID NO: 3, and a CDR1 domain comprising the amino acid sequence of SEQ ID NO: 2, and a light chain variable region comprising a CDR3 domain comprising the amino acid sequence of SEQ ID NO: 8, a CDR2 domain comprising the amino acid sequence of SEQ ID NO: 7, and a CDR1 domain comprising the amino acid sequence of SEQ ID NO: 6.

The present invention also features in other aspects, a binding protein comprising an antigen binding domain, said binding protein capable of binding a TAT protein transduction domain, said antigen binding domain comprising a heavy chain variable region comprising a CDR3 domain comprising the amino acid sequence of SEQ ID NO: 4, a CDR2 domain comprising the amino acid sequence of SEQ ID NO: 3, and a CDR1 domain comprising the amino acid sequence of SEQ ID NO: 2, and a light chain variable region comprising a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10.

The present invention also features in other aspects, a binding protein comprising an antigen binding domain, said binding protein capable of binding a TAT protein transduction domain, said antigen binding domain comprising a heavy chain variable region comprising a CDR3 domain comprising the amino acid sequence of SEQ ID NO: 18, a CDR2 domain comprising the amino acid sequence of SEQ ID NO: 17, and a CDR1 domain comprising the amino acid sequence of SEQ ID NO: 16, and a light chain variable region comprising a CDR3 domain comprising the amino acid sequence of SEQ ID NO: 22, a CDR2 domain comprising the amino acid sequence of SEQ ID NO: 21, and a CDR1 domain comprising the amino acid sequence of SEQ ID NO: 20.

In a further embodiment, the antigen binding domain comprises a heavy chain variable region comprising the amino acid sequence set forth in SEQ ID NO: 1 and a light chain variable region comprising the amino acid sequence set forth in SEQ ID NO: 5.

In another further embodiment, the antigen binding domain comprises a heavy chain variable region comprising the amino acid sequence set forth in SEQ ID NO: 1 and a light chain variable region comprising the amino acid sequence set forth in SEQ ID NO: 9.

In another further embodiment, the antigen binding domain comprises a heavy chain variable region comprising the amino acid sequence set forth in SEQ ID NO: 1 and a light chain variable region comprising the amino acid sequence set forth in SEQ ID NO: 13.

In another further embodiment, the antigen binding domain comprises a heavy chain variable region comprising the amino acid sequence set forth in SEQ ID NO: 14 and a light chain variable region comprising the amino acid sequence set forth in SEQ ID NO: 5.

In another further embodiment, the antigen binding domain comprises a heavy chain variable region comprising the amino acid sequence set forth in SEQ ID NO: 15 and a light chain variable region comprising the amino acid sequence set forth in SEQ ID NO: 19.

In certain embodiments, the term “10-1” refers to a hybridoma that produces an antibody comprising (i) one variable heavy chain having an amino acid sequence comprising SEQ ID NO: 1; and (ii) one variable light chain having an amino acid sequence comprising SEQ ID NO: 5. In certain embodiments, the 10-1 heavy chain variable region comprises a CDR3 domain comprising the amino acid sequence of SEQ ID NO: 4, a CDR2 domain comprising the amino acid sequence of SEQ ID NO: 3, and a CDR1 domain comprising the amino acid sequence of SEQ ID NO: 2, and the light chain variable region comprises a CDR3 domain comprising the amino acid sequence of SEQ ID NO: 8, a CDR2 domain comprising the amino acid sequence of SEQ ID NO: 7, and a CDR1 domain comprising the amino acid sequence of SEQ ID NO: 6. In certain embodiments, antibody 10-1 can have an on rate constant (K_{ON}) to a TAT protein transduction domain of at least about 1×10⁴ M⁻¹ s⁻¹ to about 6×10⁶ M⁻¹ s⁻¹ as measured by surface plasmon resonance. In other embodiments, the binding protein according to the present invention can have an on rate constant (K_{ON}) to a TAT protein transduction domain of least about 2.7×10⁵ M⁻¹ s⁻¹ as measured by surface plasmon resonance. In other embodiments, the binding protein according to the present invention can have a dissociation constant (K_{D}) to a TAT protein transduction domain of 1.0×10⁻¹² s⁻¹ or less. In certain preferred embodiments, the binding protein according to the present invention has a dissociation constant (K_{D}) to a TAT protein transduction domain of about 1.0×10's⁻¹ or less; 1.0×10⁻⁸ s⁻¹ or less; 1.0×10⁻⁹ s⁻¹ or less; 1.0×10⁻¹⁰ s⁻¹ or less; 1.0×10⁻¹¹ s⁻¹ or less; and 1.0×10⁻¹² s⁻¹ or less. According to preferred embodiments of the invention, the isotype of the antibody construct produced by the 10-1 hybridoma clone is IgG1κ.

In some embodiments, an anti-TAT antibody, or antigen-binding portion thereof, comprises a heavy chain comprising an amino acid sequence set forth in SEQ ID NO: 1, or a sequence having at least 90%, 95%, 96%, 97%, 98%, or 99% identity to SEQ ID NO: 1, and/or a light chain comprising an amino acid sequence set forth in SEQ ID NO: 5, or a sequence having at least 90%, 95%, 96%, 97%, 98%, or 99% identity to SEQ ID NO: 5.

In certain embodiments, the term “10-12” refers to a hybridoma that produces an antibody comprising (i) one variable heavy chain having an amino acid sequence comprising SEQ ID NO: 14; and (ii) one variable light chain having an amino acid sequence comprising SEQ ID NO: 5. In certain embodiments, the 10-12 heavy chain variable region comprises a CDR3 domain comprising the amino acid sequence of SEQ ID NO: 4, a CDR2 domain comprising the amino acid sequence of SEQ ID NO: 3, and a CDR1 domain comprising the amino acid sequence of SEQ ID NO: 2, and the light chain variable region comprises a CDR3 domain comprising the amino acid sequence of SEQ ID NO: 8, a CDR2 domain comprising the amino acid sequence of SEQ ID NO: 7, and a CDR1 domain comprising the amino acid sequence of SEQ ID NO: 6. In certain embodiments, antibody 10-12 can have an on rate constant (K_{ON}) to a TAT protein transduction domain of at least about 1×10⁴ M⁻¹ s⁻¹ to about 6×10⁶M⁻¹ s⁻¹ as measured by surface plasmon resonance. In other embodiments, the binding protein according to the present invention can have an on rate constant (K_{ON}) to a TAT protein transduction domain of least about 2.7×10⁵M⁻¹ s⁻¹ as measured by surface plasmon resonance. In other embodiments, the binding protein according to the present invention can have a dissociation constant (K_{D}) to a TAT protein transduction domain of 1.0×10⁻¹² s⁻¹ or less. In certain preferred embodiments, the binding protein according to the present invention has a dissociation constant (K_{D}) to a TAT protein transduction domain of about 1.0×10⁻⁷ s⁻¹ or less; 1.0×10⁻⁸ s⁻¹ or less; 1.0×10⁻⁹ s⁻¹ or less; 1.0×10⁻¹⁰ s⁻¹ or less; 1.0×10¹¹ s⁻¹ or less; and 1.0×10⁻¹² s⁻¹ or less. According to preferred embodiments of the invention, the isotype of the antibody construct produced by the 10-12 hybridoma clone is IgG1/κ.

In some embodiments, an anti-TAT antibody, or antigen-binding portion thereof, comprises a heavy chain comprising an amino acid sequence set forth in SEQ ID NO: 14, or a sequence having at least 90%, 95%, 96%, 97%, 98%, or 99% identity to SEQ ID NO: 14, and/or a light chain comprising an amino acid sequence set forth in SEQ ID NO: 5, or a sequence having at least 90%, 95%, 96%, 97%, 98%, or 99% identity to SEQ ID NO: 5.

In certain embodiments, the terms “10-4,” “10-5”, “12-1”, and “12-3” refer to hybridomas that produce an antibody comprising (i) one variable heavy chain having an amino acid sequence comprising SEQ ID NO: 1; and (ii) one variable light chain having an amino acid sequence comprising SEQ ID NO: 9. In certain embodiments, the 10-4, 10-5, 12-1, and 12-3 heavy chain variable regions comprise a CDR3 domain comprising the amino acid sequence of SEQ ID NO: 4, a CDR2 domain comprising the amino acid sequence of SEQ ID NO: 3, and a CDR1 domain comprising the amino acid sequence of SEQ ID NO: 2, and the light chain variable regions comprise a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10. In certain embodiments, antibodies 10-4, 10-5, 12-1, and 12-3 can have an on rate constant (K_{ON}) to a TAT protein transduction domain of at least about 1×10⁴ M⁻¹ s⁻¹ to about 6×10⁶M⁻¹ s⁻¹ as measured by surface plasmon resonance. In other embodiments, the binding protein according to the present invention can have an on rate constant (K_{ON}) to a TAT protein transduction domain of least about 2.7×10⁵M⁻¹ s⁻¹ as measured by surface plasmon resonance. In other embodiments, the binding protein according to the present invention can have a dissociation constant (Ku) to a TAT protein transduction domain of 1.0×10⁻¹² s⁻¹ or less. In certain preferred embodiments, the binding protein according to the present invention has a dissociation constant (Ku) to a TAT protein transduction domain of about 1.0×10⁻⁷ s⁻¹ or less; 1.0×10⁻⁸ s⁻¹ or less; 1.0×10⁻⁹ s⁻¹ or less; 1.0×10⁻¹⁰ s⁻¹ or less; 1.0×10⁻¹¹ s⁻¹ or less; and 1.0×10⁻¹² s⁻¹ or less. According to preferred embodiments of the invention, the isotype of the antibody construct produced by the 10-4, 10-5, 12-1, and 12-3 hybridoma clones is IgG₁/κ.

In some embodiments, an anti-TAT antibody, or antigen-binding portion thereof, comprises a heavy chain comprising an amino acid sequence set forth in SEQ ID NO: 1, or a sequence having at least 90%, 95%, 96%, 97%, 98%, or 99% identity to SEQ ID NO: 1, and/or a light chain comprising an amino acid sequence set forth in SEQ ID NO: 9, or a sequence having at least 90%, 95%, 96%, 97%, 98%, or 99% identity to SEQ ID NO: 9.

In certain embodiments, the terms “10-9,” “12-8”, and “12-10” refer to hybridomas that produce an antibody comprising (i) one variable heavy chain having an amino acid sequence comprising SEQ ID NO: 1; and (ii) one variable light chain having an amino acid sequence comprising SEQ ID NO: 13. In certain embodiments, the 10-9, 12-8, and 12-10 heavy chain variable regions comprise a CDR3 domain comprising the amino acid sequence of SEQ ID NO: 4, a CDR2 domain comprising the amino acid sequence of SEQ ID NO: 3, and a CDR1 domain comprising the amino acid sequence of SEQ ID NO: 2, and the light chain variable regions comprise a CDR3 domain comprising the amino acid sequence of SEQ ID NO: 8, a CDR2 domain comprising the amino acid sequence of SEQ ID NO: 7, and a CDR1 domain comprising the amino acid sequence of SEQ ID NO: 6. In certain embodiments, antibodies 10-9, 12-8, and 12-10 can have an on rate constant (K_{ON}) to a TAT protein transduction domain of at least about 1×10⁴ M⁻¹ s⁻¹ to about 6×10⁶ M⁻¹ s⁻¹ as measured by surface plasmon resonance. In other embodiments, the binding protein according to the present invention can have an on rate constant (K_{ON}) to a TAT protein transduction domain of least about 2.7×10⁵ M⁻¹ s⁻¹ as measured by surface plasmon resonance. In other embodiments, the binding protein according to the present invention can have a dissociation constant (Ku) to a TAT protein transduction domain of 1.0×10⁻¹² s⁻¹ or less. In certain preferred embodiments, the binding protein according to the present invention has a dissociation constant (Ku) to a TAT protein transduction domain of about 1.0×10⁻⁷ s⁻¹ or less; 1.0×10⁻⁸ s⁻¹ or less; 1.0×10⁻⁹ s⁻¹ or less; 1.0×10⁻¹⁰ s⁻¹ or less; 1.0×10⁻¹¹ s⁻¹ or less; and 1.0×10⁻¹² s⁻¹ or less. According to preferred embodiments of the invention, the isotype of the antibody construct produced by the 10-9, 12-8, and 12-10 hybridoma clones is IgG1/κ.

In some embodiments, an anti-TAT antibody, or antigen-binding portion thereof, comprises a heavy chain comprising an amino acid sequence set forth in SEQ ID NO: 1, or a sequence having at least 90%, 95%, 96%, 97%, 98%, or 99% identity to SEQ ID NO: 1, and/or a light chain comprising an amino acid sequence set forth in SEQ ID NO: 13, or a sequence having at least 90%, 95%, 96%, 97%, 98%, or 99% identity to SEQ ID NO: 13.

In certain embodiments, the term “6.3” refers to a hybridoma that produces an antibody comprising (i) one variable heavy chain having an amino acid sequence comprising SEQ ID NO: 14; and (ii) one variable light chain having an amino acid sequence comprising SEQ ID NO: 5. In certain embodiments, the 6.3 heavy chain variable region comprises a CDR3 domain comprising the amino acid sequence of SEQ ID NO: 4, a CDR2 domain comprising the amino acid sequence of SEQ ID NO: 3, and a CDR1 domain comprising the amino acid sequence of SEQ ID NO: 2, and the light chain variable region comprises a CDR3 domain comprising the amino acid sequence of SEQ ID NO: 8, a CDR2 domain comprising the amino acid sequence of SEQ ID NO: 7, and a CDR1 domain comprising the amino acid sequence of SEQ ID NO: 6. In certain embodiments, antibody 6.3 can have an on rate constant (K_{ON}) to a TAT protein transduction domain of at least about 1×10⁴ M⁻¹ s⁻¹ to about 6×10⁶M⁻¹ s⁻¹ as measured by surface plasmon resonance. In other embodiments, the binding protein according to the present invention can have an on rate constant (K_{ON}) to a TAT protein transduction domain of least about 2.7×10⁵ M⁻¹ s⁻¹ as measured by surface plasmon resonance. In other embodiments, the binding protein according to the present invention can have a dissociation constant (K_{D}) to a TAT protein transduction domain of 1.0×10⁻¹² s⁻¹ or less. In certain preferred embodiments, the binding protein according to the present invention has a dissociation constant (K_{D}) to a TAT protein transduction domain of about 1.0×10⁻⁷ s⁻¹ or less; 1.0×10⁻⁸ s⁻¹ or less; 1.0×10⁻⁹ s⁻¹ or less; 1.0×10⁻¹⁰ s⁻¹ or less; 1.0×10⁻¹¹ s⁻¹ or less; and 1.0×10⁻¹² s⁻¹ or less. According to preferred embodiments of the invention, the isotype of the antibody construct produced by the 6.3 hybridoma clone is IgG21κ.

In some embodiments, an anti-TAT antibody, or antigen-binding portion thereof, comprises a heavy chain comprising an amino acid sequence set forth in SEQ ID NO: 15, or a sequence having at least 90%, 95%, 96%, 97%, 98%, or 99% identity to SEQ ID NO: 15, and/or a light chain comprising an amino acid sequence set forth in SEQ ID NO: 19, or a sequence having at least 90%, 95%, 96%, 97%, 98%, or 99% identity to SEQ ID NO: 19.

According to preferred embodiments of the present invention, the binding protein as described herein is an antibody.

Accordingly, the present invention features an antibody construct comprising a binding protein as described herein, wherein the antibody construct further comprises a linker polypeptide or an immunoglobulin constant domain.

The antibody construct according the present invention may comprise a heavy chain immunoglobulin constant domain selected from the group consisting of a IgM constant domain, a IgG4 constant domain, a IgG1 constant domain, a IgE constant domain, a IgG2 constant domain, a IgG3 constant domain and a IgA constant domain.

Furthermore, the antibody can comprise a light chain constant region, either a kappa light chain constant region or a lambda light chain constant region.

In certain embodiments, the binding protein according to the present invention can have an on rate constant (Kon) to a TAT protein transduction domain selected from the group consisting of about 1×10⁴ M⁻¹ s⁻¹ to about 6×10⁶ M⁻¹ s⁻¹ as measured by surface plasmon resonance. In other embodiments, the binding protein according to the present invention can have an on rate constant (K_{ON}) to TAT peptide of least about 2.5×10⁵M⁻¹ s⁻¹ and at least about 2.7×10⁵M⁻¹ s⁻¹, as measured by surface plasmon resonance.

In other embodiments, the binding protein according to the present invention can have a dissociation constant (K_{D}) to a TAT protein transduction domain selected from the group consisting of 1.0×10⁻⁷ s⁻¹ or less; 1.0×10⁻⁸ s⁻¹ or less; 1.0×10⁻⁹ s⁻¹ or less; 1.0×10⁻¹⁰ s⁻¹ or less; 1.0×10⁻¹¹ s⁻¹ or less; and 1.0×10⁻¹² s⁻¹ or less. In certain preferred embodiments, the binding protein according to the present invention has a dissociation constant (K_{D}) to TAT peptide of 1.0×10⁻⁷ s⁻¹ or less.

The binding protein can be selected from an immunoglobulin molecule, a monoclonal antibody, a murine antibody, a chimeric antibody, a CDR-grafted antibody, a humanized antibody, a single domain antibody, a Fv, a disulfide linked Fv, a scFv, a diabody, a Fab, a Fab′, a F(ab′)2, a multispecific antibody, a dual specific antibody, and a bispecific antibody.

Alternatively, the antibody portion can be, for example, a Fab fragment or a single chain Fv fragment.

Replacements of amino acid residues in the Fc portion to alter antibody effector function are known in the art (Winter, et al. U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG₁ and IgG₃, mediate ADCC and CDC via binding to FcγRs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment provides a labeled binding protein wherein an antibody or antibody portion of the invention is derivatized or linked to one or more functional molecule(s) or cargo molecule(s). In one embodiment, the cargo moiety is another peptide or protein, e.g., a frataxin polypeptide. In one embodiment, the cargo moiety is a polypeptide selected from the group consisting of Abarelix, Abatacept, Abciximab, Adalimumab, Aflibercept, Agalsidase beta, Albiglutide, Aldesleukin, Alefacept, Alemtuzumab, Alemtuzumab, Alglucerase, Alglucosidase alfa, Alirocumab, Aliskiren, Alpha-1-proteinase inhibitor, Alteplase, Anakinra, Ancestim, Anistreplase, Anthrax immune globulin human, Antihemophilic Factor, Anti-inhibitor coagulant complex, Antithrombin Alfa, Antithrombin III human, Antithymocyte globulin, Anti-thymocyte Globulin (Equine), Anti-thymocyte Globulin (Rabbit), Aprotinin, Arcitumomab, Asfotase Alfa, Asparaginase, Asparaginase Erwinia Chrysanthemi, Atezolizumab, Autologous cultured chondrocytes, Basiliximab, Becaplermin, Belatacept, Belimumab, Beractant, Bevacizumab, Bivalirudin, Blinatumomab, Botulinum Toxin Type A, Botulinum Toxin Type B, Brentuximab Vedotin, Brodalumab, Buserelin, C1 Esterase Inhibitor (Human), C1 Esterase Inhibitor (Recombinant), Canakinumab, Canakinumab, Capromab, Certolizumab Pegol, Cetuximab, Choriogonadotropin alfa, Chorionic Gonadotropin (Human), Chorionic Gonadotropin (Recombinant), Coagulation factor IX, Coagulation factor VIIa, Coagulation factor X human, Coagulation Factor XIII A-Subunit (Recombinant), Collagenase, Conestat alfa, Corticotropin, Cosyntropin, Daclizumab, Daptomycin, Daratumumab, Darbepoetin alfa, Defibrotide, Denileukin diftitox, Denosumab, Desirudin, Digoxin Immune Fab (Ovine), Dinutuximab, Dornase alfa, Drotrecogin alfa, Dulaglutide, Eculizumab, Efalizumab, Efmoroctocog alfa, Elosulfase alfa, Elotuzumab, Enfuvirtide, Epoetin alfa, Epoetin zeta, Eptifibatide, Etanercept, Evolocumab, Exenatide, Factor IX Complex (Human), Fibrinogen Concentrate (Human), Fibrinolysin(Plasmin), Filgrastim, Filgrastim-sndz, Follitropin alpha, Follitropin beta, Galsulfase, Gastric intrinsic factor, Gemtuzumab ozogamicin, Glatiramer acetate, Glucagon recombinant, Glucarpidase, Golimumab, Gramicidin D, Hepatitis A Vaccine, Hepatitis B immune globulin, Human calcitonin, Human Clostridium tetani toxoid immune globulin, Human rabies virus immune globulin, Human Rho(D) immune globulin, Human Serum Albumin, Human Varicella-Zoster Immune Globulin, Hyaluronidase, Hyaluronidase (Human Recombinant), Ibritumomab, Ibritumomab tiuxetan, Idarucizumab, Idursulfase, Imiglucerase, Immune Globulin Human, Infliximab, Insulin Aspart, Insulin, Gemtuzumab ozogamicin, Glatiramer acetate, Glucagon recombinant, Glucarpidase, Golimumab, Gramicidin D, Hepatitis A Vaccine, Hepatitis B immune globulin, Human calcitonin, Human Clostridium tetani toxoid immune globulin, Human rabies virus immune globulin, Human Rho(D) immune globulin, Human Serum Albumin, Human Varicella-Zoster Immune Globulin, Hyaluronidase, Hyaluronidase (Human Recombinant), Ibritumomab, Ibritumomab tiuxetan, Idarucizumab, Idursulfase, Imiglucerase, Immune Globulin Human, Infliximab, Insulin aspart, Insulin Beef, Insulin Degludec, Insulin detemir, Insulin Glargine, Insulin glulisine, Insulin Lispro, Insulin (Pork), Insulin Regular, Insulin (porcine), Insulin-isophane, Interferon Alfa-2a, Recombinant, Interferon alfa-2b, Interferon alfacon-1, Interferon alfa-n1, Interferon alfa-n3, Interferon beta-1a, Interferon beta-1b, Interferon gamma-1b, Intravenous Immunoglobulin, Ipilimumab, Ixekizumab, Laronidase, Lenograstim, Lepirudin, Leuprolide, Liraglutide, Lucinactant, Lutropin alfa, Mecasermin, Menotropins, Mepolizumab, Methoxy polyethylene glycol-epoetin beta, Metreleptin, Muromonab, Natalizumab, Natural alpha interferon OR multiferon, Necitumumab, Nesiritide, Nivolumab, Obiltoxaximab, Obinutuzumab, Ocriplasmin, Ofatumumab, Omalizumab, Oprelvekin, OspA lipoprotein, Oxytocin, Palifermin, Palivizumab, Pancrelipase, Panitumumab, Pegademase bovine, Pegaptanib, Pegaspargase, Pegfilgrastim, Peginterferon alfa-2a, Peginterferon alfa-2b, Peginterferon beta-1a, Pegloticase, Pegvisomant, Pembrolizumab, Pertuzumab, Poractant alfa, Pramlintide, Preotact, Protamine sulfate, Protein S human, Prothrombin complex concentrate, Ragweed Pollen Extract, Ramucirumab, Ranibizumab, Rasburicase, Raxibacumab, Reteplase, Rilonacept, Rituximab, Romiplostim, Sacrosidase, Salmon Calcitonin, Sargramostim, Satumomab Pendetide, Sebelipase alfa, Secretin, Secukinumab, Sermorelin, Serum albumin, Serum albumin iodonated, Siltuximab, Simoctocog Alfa, Sipuleucel-T, Somatotropin Recombinant, Somatropin recombinant, Streptokinase, Sulodexide, Susoctocog alfa, Taliglucerase alfa, Teduglutide, Teicoplanin, Tenecteplase, Teriparatide, Tesamorelin, Thrombomodulin Alfa, Thymalfasin, Thyroglobulin, Thyrotropin Alfa, Tocilizumab, Tositumomab, Trastuzumab, Tuberculin Purified Protein Derivative, Turoctocog alfa, Urofollitropin, Urokinase, Ustekinumab, Vasopressin, Vedolizumab, Velaglucerase alfa, Thrombomodulin Alfa, Thymalfasin, Thyroglobulin, Thyrotropin Alfa, Tocilizumab, Tositumomab, Trastuzumab, Tuberculin Purified Protein Derivative, Turoctocog alfa, Urofollitropin, Urokinase, Ustekinumab, Vasopressin, Vedolizumab, Velaglucerase alfa.

In another embodiment, the protein is selected from those included in Raghava, Gajendra P. S.; Usmani, Salman Sadullah; Bedi, Gursimran; Samuel, Jesse S.; Singh, Sandeep; Kalra, Sourav; et al. (2017): THPdb: Database of FDA Approved Peptide and Protein Therapeutics, the contents of which are hereby incorporated herein by reference.

In another embodiment, the cargo moiety is a pharmacologically active compound, a small molecule, a liposome enclosing protein, a radionuclide or radionuclide labeled compound, a nucleic acid, e.g., an siRNA, shRNA, miRNA, phosphorothioate modified RNA, aptamer, a phosphorodiamidate morpholino oligomer (PMO), or any combination thereof. For example, a labeled binding protein of the invention can be derived by functionally linking an antibody or antibody portion of the invention (by chemical coupling, genetic fusion, noncovalent association or otherwise) to one or more other molecular entities, such as another antibody (e.g., a bispecific antibody or a diabody), a detectable agent, a pharmaceutical agent, a protein or peptide that can mediate the association of the antibody or antibody portion with another molecule (such as a streptavidin core region or a polyhistidine tag), and/or a cytotoxic or therapeutic agent selected from the group consisting of a mitotic inhibitor, an antitumor antibiotic, an immunomodulating agent, a vector for gene therapy, an alkylating agent, an antiangiogenic agent, an antimetabolite, a boron-containing agent, a chemoprotective agent, a hormone, an antihormone agent, a corticosteroid, a photoactive therapeutic agent, an oligonucleotide, a radionuclide agent, a topoisomerase inhibitor, a tyrosine kinase inhibitor, a radiosensitizer, and a combination thereof.

The binding protein of the present invention can be immobilized on a solid support. Solid supports are known in the art, and include, for example, a plate, a bead, or a chromatography resin, such as, for example, trisacryl, sepharose, agarose, polyacrylamide, poros, poroshell, captol, toyopearl, hypercel, cellulosic types, sephadex (dextrans), amberlite, amberchrome, amberjet, dowex, optipore, supelpak, combigel, monosphere, duolite, diaion, aminolink, sulfolink, carboxylink, glycolink, marathon, or glycocatch. In one embodiment, the bead or chromatography resin comprises protein A agarose or protein G agarose.

In one embodiment, the antibody is conjugated to an imaging agent or a detection molecule or label (used interchangeably herein). Examples of imaging agents that may be used in the compositions and methods described herein include, but are not limited to, a radiolabel (e.g., indium), an enzyme, e.g., horseradish peroxidase, a fluorescent label, a luminescent label, a bioluminescent label, a magnetic label, biotin, SULFO-TAG labeled Streptavidin, alkaline phosphatase, cresol violet, quantum dots, fluorescein isothiocyanate (FITC), infrared molecule, or an electron paramagnetic resonance (EPR) spin tracer label.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like.

An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

In still another embodiment, the glycosylation of the antibody or antigen-binding portion of the invention is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in PCT Publication WO2003016466A2, and U.S. Pat. Nos. 5,714,350 and 6,350,861, each of which is incorporated herein by reference in its entirety.

Additionally or alternatively, a modified antibody of the invention can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342 80, each of which is incorporated herein by reference in its entirety.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. Preferably the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. patent Publication Nos. 20040018590 and 20020137134 and PCT publication WO2005100584 A2).

In addition to the binding proteins, the present invention is also directed to an anti-idiotypic (anti-Id) antibody specific for such binding proteins of the invention. An anti-Id antibody is an antibody, which recognizes unique determinants generally associated with the antigen-binding region of another antibody. The anti-Id can be prepared by immunizing an animal with the binding protein or a CDR containing region thereof. The immunized animal will recognize, and respond to the idiotypic determinants of the immunizing antibody and produce an anti-Id antibody. The anti-Id antibody may also be used as an “immunogen” to induce an immune response in yet another animal, producing a so-called anti-anti-Id antibody.

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. Preferably, the protein having a particularly selected novel glycosylation pattern exhibits improved or altered biological properties.

Antibodies may be produced by any of a number of techniques. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies disclosed herein include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the disclosure. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure and the other heavy and light chain are specific for an antigen other than the antigens of interest by crosslinking an antibody of the disclosure to a second antibody by standard chemical crosslinking methods.

In a preferred system for recombinant expression of an antibody, or antigen binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the disclosure provides a method of synthesizing a recombinant antibody by culturing a host cell in a suitable culture medium until a recombinant antibody is synthesized. Recombinant antibodies may be produced using nucleic acid molecules corresponding to the amino acid sequences disclosed herein. In one embodiment, the nucleic acid molecules set forth in SEQ ID NOs: 25-45 (see Table 2, below) are used in the production of a recombinant antibody. The method can further comprise isolating the recombinant antibody from the culture medium.

B. Methods of Making Anti-TAT Peptide Antibodies

Antibodies of the present invention may be made by any of a number of techniques known in the art.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammering et al., “Monoclonal Antibodies And T cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; and 4,493,890. Methods for producing polyclonal antibodies are well-known in the art. See U.S. Pat. No. 4,493,795 to Nestor et al.

Panels of monoclonal antibodies produced against a TAT peptide, e.g., a TAT transduction domain, can be screened for various properties; i.e., isotype, epitope, affinity, etc.

A monoclonal antibody, typically containing Fab and/or F (ab′)2 portions of useful antibody molecules, can be prepared using the hybridoma technology described in Antibodies-A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with an appropriate TAT peptide, e.g., a TAT transduction domain.

Splenocytes are typically fused with myeloma cells using polyethylene glycol (PEG) 6000. Fused hybrids are selected by their sensitivity to HAT. Hybridomas producing a monoclonal antibody useful in practicing this invention are identified by their ability to immunoreact with the present antibody or binding member and their ability to inhibit specified tumorigenic or hyperproliferative activity in target cells.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Media useful for the preparation of these compositions are both well-known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/1 glucose, 20 mm glutamine, and 20% fetal calf serum. An exemplary inbred mouse strain is the Balb/c.

1. Anti-TAT Peptide Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. For example, monoclonal antibodies can be generated by the method of culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention. Briefly, mice can be immunized with a TAT peptide antigen, e.g., a TAT transduction domain antigen. In certain embodiments, the TAT peptide antigen, e.g., the TAT transduction domain antigen, is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with a TAT peptide antigen, antibodies and/or antibody-producing cells may be obtained from the animal. An anti-TAT peptide antibody-containing serum is obtained from the animal by bleeding or sacrificing the animal. The serum may be used as it is obtained from the animal, an immunoglobulin fraction may be obtained from the serum, or the anti-TAT peptide antibodies may be purified from the serum. Serum or immunoglobulins obtained in this manner are polyclonal, thus having a heterogeneous array of properties.

Once an immune response is detected, e.g., antibodies specific for the antigen TAT peptide, e.g., the TAT transduction domain antigen, are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding TAT peptide, e.g., a TAT transduction domain. Ascites fluid, which generally contains high levels of antibodies, can be generated by immunizing mice with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using TAT peptide, or a portion thereof, or a cell expressing TAT peptide, or a portion thereof. The initial screening is performed using an enzyme-linked immunoassay (ELISA) or a radioimmunoassay (RIA). An example of ELISA screening is provided in WO 00/37504, herein incorporated by reference.

Anti-TAT peptide antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the hybridomas are mouse hybridomas, as described herein. In another preferred embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an anti-TAT peptide antibody.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

2. Anti-TAT Peptide Monoclonal Antibodies Using Selected Lymphocyte Antibody Method

In another aspect of the invention, recombinant antibodies are generated from single, isolated lymphocytes using a procedure referred to in the art as the selected lymphocyte antibody method (SLAM), as described in U.S. Pat. No. 5,627,052, PCT Publication WO 92/02551 and Babcock, J. S. et al. (1996) Proc. Natl. Acad. Sci. USA 93:7843-7848. In this method, single cells secreting antibodies of interest, e.g., lymphocytes derived from any one of the immunized animals described above, are screened using an antigen-specific hemolytic plaque assay, wherein the antigen TAT peptide, a subunit of TAT peptide, e.g., a TAT transduction domain antigen, or a fragment thereof, is coupled to sheep red blood cells using a linker, such as biotin, and used to identify single cells that secrete antibodies with specificity for TAT peptide, e.g., a TAT transduction domain. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to TAT peptide, e.g., a TAT transduction domain. The amplified immunoglobulin sequences further can be manipulated in vitro, such as by in vitro affinity maturation methods such as those described in PCT Publication WO 97/29131 and PCT Publication WO 00/56772.

3. Anti-TAT Peptide Monoclonal Antibodies Using Recombinant Antibody Libraries

In vitro methods also can be used to make the antibodies of the invention, wherein an antibody library is screened to identify an antibody having the desired binding specificity. Methods for such screening of recombinant antibody libraries are well known in the art and include methods described in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mot Blot 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, US patent application publication 20030186374, and PCT Publication No. WO 97/29131, the contents of each of which are incorporated herein by reference.

The recombinant antibody library may be from a subject immunized with TAT peptide, or a portion of TAT peptide, e.g., a TAT transduction domain. Alternatively, the recombinant antibody library may be from a naïve subject, i.e., one who has not been immunized with TAT peptide, such as a human antibody library from a human subject who has not been immunized with TAT peptide. Antibodies of the invention are selected by screening the recombinant antibody library with the peptide comprising TAT peptide to thereby select those antibodies that recognize TAT peptide, e.g., TAT transduction domain. Methods for conducting such screening and selection are well known in the art, such as described in the references in the preceding paragraph. To select antibodies of the invention having particular binding affinities for TAT peptide, e.g., a TAT transduction domain, such as those that dissociate from TAT transduction domain with a particular k_{off} rate constant, the art-known method of surface plasmon resonance can be used to select antibodies having the desired k_{off} rate constant. To select antibodies of the invention having a particular neutralizing activity for a TAT transduction domain, such as those with a particular an IC₅₀, standard methods known in the art for assessing the inhibition of TAT peptide activity may be used.

In one aspect, the invention pertains to an isolated antibody, or an antigen-binding portion thereof, that binds TAT peptide, in particular human TAT peptide. In various embodiments, the antibody is a recombinant antibody or a monoclonal antibody.

For example, the antibodies of the present invention can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

Alternative to screening of recombinant antibody libraries by phage display, other methodologies known in the art for screening large combinatorial libraries can be applied to the identification of dual specificity antibodies of the invention. One type of alternative expression system is one in which the recombinant antibody library is expressed as RNA-protein fusions, as described in PCT Publication No. WO 98/31700 by Szostak and Roberts, and in Roberts, R. W. and Szostak, J. W. (1997) Proc. Natl. Acad. Sci. USA 94:12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above.

In another approach the antibodies of the present invention can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies of the present invention include those disclosed in Wittrup et al. (U.S. Pat. No. 6,699,658) incorporated herein by reference.

4. Recombinant Anti-TAT Peptide Antibodies

Antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the invention in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

The invention features in certain embodiments an isolated nucleic acid encoding a binding protein amino acid sequence as described herein. The invention also features in other certain embodiments, an isolated nucleic acid encoding an antibody construct amino acid sequence as described herein. In methods of production, an expression vector comprises the isolated nucleic acid. Non-limiting examples of such expression vectors are the pUC series of vectors (Fermentas Life Sciences), the pBluescript series of vectors (Stratagene, La Jolla, Calif.), the pET series of vectors (Novagen, Madison, Wis.), the pGEX series of vectors (Pharmacia Biotech, Uppsala, Sweden), and the pEX series vectors (Clontech, Palo Alto, Calif.).

A host cell comprises the vector described herein. According to embodiments of the invention, the host cell is a prokaryotic cell or a eukaryotic cell. For example, the prokaryotic host cells is E. coli. The eukaryotic cell may be selected from a protist cell, an animal cell, a plant cell or a fungal cell. The animal cell may be selected from a mammalian cell, an avian cell, and an insect cell. Preferably, the host cell is selected from a CHO cell, a COS cell, a yeast cell, and an insect Sf9 cell. In further related embodiments, the yeast cell is Saccharomyces cerevisiae.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell of the invention in a suitable culture medium until a recombinant antibody of the invention is synthesized. The method can further comprise isolating the recombinant antibody from the culture medium.

Also contemplated by the present invention are various methods of production of a protein capable of binding TAT peptide or of production of an antibody, or antigen binding portion thereof that binds TAT peptide, comprising culturing a host cell as described herein in culture medium so that the nucleic acid is expressed and the antibody is produced. An exemplary method of producing a protein capable of binding TAT peptide comprises culturing a host cell as described herein in culture medium under conditions sufficient to produce a binding protein capable of binding TAT peptide.

The invention also features a protein produced according to said methods.

5. Humanized Anti TAT Peptide Antibodies

Humanized antibodies are antibody molecules from non-human species antibody that binds the desired antigen having one or more complementarity determining regions (CDRs) from the non-human species and framework regions from a human immunoglobulin molecule. Known human Ig sequences are disclosed, e.g., at the following: ncbi.nlm.nih.gov/entrez-/query.fcgi; atcc.org/phage/hdb.html; sciquest.com/; abcam.com/; antibodyresource.com/onlinecomp.html; public.iastate.edu/.about.pedro/research_tools.html; mgen.uni-heidelberg.de/SD/IT/IT.html; whfreeman.com/immunology/CH-05/kuby05.htm; library.thinkquest.org/12429/Immune/Antibody.html; hhmi.org/grants/lectures/1996/vlab/; path.cam.ac.ukiabout.mrc7/m-ikeimages.html; antibodyresource.com/; mcb.harvard.edu/BioLinks/Immuno-logy, iimmunologylink.com/; pathbox.wustl.edu/.about.hcenter/index.-html; biotech.ufl.edu/.about.hcl/; pebio.com/pa/340913/340913.html-; nal.usda.gov/awic/pubs/antibody/; m.ehime-u.acjp/.about.yasuhito-/Elisa.html; biodesign.com/table.asp; icnet.uk/axp/facs/davies/lin-ks.html; biotech.ufl.edu/.about.fccl/protocol.html; isac-net.org/sites_geo.html; aximtl.imt.uni-marburg.de/.about.rek/AEP-Start.html; baserv.uci.kun.nl/about.jraats/linksl.html; recab.uni-hd.de/immuno.bme.nwu.edu/; mrc-cpe.cam.ac.uk/imt-doc/pu-blic/INTRO.html; ibt.unam.mx/vir/V_mice.html; imgt.cnusc.fr:8104/; biochem.ucl.ac.uk/.about.martin/abs/index.html; antibody.bath.ac.uk/; abgen.cvm.tamu.edu/lab/wwwabgen.html; unizh.ch/.about.honegger/AHOsem-inar/Slide01.html; cryst.bbk.ac.uk/.about.ubcg07s/; nimr.mrc.ac.uk/CC/ccaewg/ccaewg.htm; path.cam.ac.uk/.about.mrc7/h-umanisation/TAHHP.html; ibt.unam.mx/vir/structure/stat aim.html; biosci.missouri.edu/smithgp/index.html; cryst.bioc.cam.ac.ukiabo-ut.fmolina/Web-pages/Pept/spottech.html; jerini.de/fr roducts.htm; patents.ibm.com/ibm.html.Kabat et al., Sequences of Proteins of Immunological Interest, U.S. Dept. Health (1983), each entirely incorporated herein by reference. Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al., Nature 321:522 (1986); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994); Roguska. et al., PNAS 91:969-973 (1994); PCT publication WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766886, 5,714,352, 6,204,023, 6,180,370, 5,693,762, 5,530,101, 5,585,089, 5,225,539; 4,816,567, each entirely incorporated herein by reference, included references cited therein.

Humanized anti-TAT peptide antibodies are contemplated by the present invention.

### III. Uses of Anti-TAT Peptide Binding Proteins

A. Detection of TAT Peptide Including TAT Fusion Molecules

Given their ability to bind to a TAT protein transduction domain, the anti-TAT peptide binding proteins, e.g., antibodies, or portions thereof, of the invention can be used to detect TAT peptide (e.g., in a biological sample, such as serum, plasma, urine, tissues or cells), using a conventional immunoassay, such as an enzyme linked immunosorbent assays (ELISA), an radioimmunoassay (MA), antibody-labeled fluorescence imaging, or tissue immunohistochemistry. It is understood that the invention includes the detection of any fragments of TAT peptide polypeptide as long as the fragment allows for the specific identification of TAT peptide by a detection method of the invention. In a specific embodiment, the TAT peptide binding proteins of the invention specifically bind to a TAT protein transduction domain. For example, an ELISA antibody must be able to bind to the TAT peptide fragment, e.g., the TAT protein transduction domain, so that detection is possible.

In another embodiment, the TAT binding proteins of the invention, e.g., antibodies, can be used to detect transcellular delivery systems, e.g., TAT-liposomes or TAT-nanoparticles, e.g., used for delivery of small molecules.

In one embodiment, the TAT binding proteins of the invention, e.g., antibodies, can be used to detect TAT peptide that is part of an intact TAT fusion molecule. For example, the TAT binding proteins of the invention can be used to detect an intact TAT fusion molecule comprising a TAT protein transduction domain linked to a cargo moiety. In one embodiment, the TAT binding protein, e.g., antibody, specifically binds to the TAT peptide of the intact TAT fusion molecule, e.g., the TAT protein transduction domain, and does not bind to the cargo moiety of the TAT fusion molecule.

In one embodiment, the cargo moiety is a polypeptide. In one embodiment, the polypeptide is frataxin, e.g., the TAT fusion molecule is a TAT-frataxin fusion molecule. In other embodiments, the cargo moiety is a nucleic acid molecule, e.g., DNA, mRNA, siRNA, shRNA.

In some embodiments, the TAT binding proteins of the invention, e.g., antibodies, can be used to detect, measure, verify delivery or quantify a TAT peptide, e.g., a TAT fusion molecule comprising a TAT protein transduction domain, in vitro or in vivo. Thus, the binding proteins of the invention are capable of detecting, quantifying, verifying delivery, and/or measuring a cargo moiety, e.g., a pro-drug or drug, conjugated to a TAT peptide, e.g., TAT fusion molecule comprising a TAT protein transduction domain, either in vivo or in vitro, for example in a particular tissue.

In some embodiments, the TAT binding proteins of the invention, e.g., antibodies, can be used to image tissue by, for example, perfusion of tissue with labelled anti-TAT binding protein. In some embodiments, the TAT binding proteins of the invention, e.g., antibodies, can be used to detect, quantify, and monitor or trace the pharmokinetics, delivery, and/or localization of a TAT peptide, e.g., a TAT fusion molecule comprising a TAT protein transduction domain, by labeling the antibody with, for example, infrared conjugates, radiolabels, or electron paramagnetic resonance (EPR) spin tracer labelling, as described herein.

In one aspect, the methods of the invention provide methods for detecting and/or quantifying the level of a TAT fusion molecule in a sample, e.g., a biological sample, comprising contacting the sample with a binding protein of the invention under conditions such that the binding protein binds to TAT protein transduction domain in the sample, to thereby detect and/or quantify the level of the TAT fusion molecule in the sample. In one embodiment, the biological sample is a liquid (e.g., blood) sample or a tissue sample.

In another aspect, the methods of the invention provide methods for detecting and/or quantifying the level of a TAT fusion molecule in vivo, comprising contacting the sample with a binding protein of the invention under conditions such that the binding protein binds to TAT protein transduction domain, and imaging the binding protein to thereby detect and/or quantify the level of the TAT fusion molecule in vivo.

In some embodiments, the TAT fusion molecule comprises a TAT protein transduction domain covalently linked to a cargo moiety. In some embodiments, the cargo moiety is a polypeptide. In some embodiments, the cargo moiety is a frataxin polypeptide.

In another embodiment, the cargo moiety is a pharmacologically active compound, a small molecule, a liposome enclosing protein, a radionuclide or radionuclide labeled compound, a nucleic acid, e.g., an siRNA, shRNA, miRNA, phosphorothioate modified RNA, aptamer, a phosphorodiamidate morpholino oligomer (PMO), or any combination thereof.

In some embodiments, the methods of the invention further comprise using the binding proteins of the invention to assess the stability of the TAT fusion molecule by, e.g., detecting the TAT fusion molecule as described herein and measuring the stability of the TAT fusion molecule over time.

In another aspect, the methods of the invention provide methods for isolating and/or purifying a TAT fusion molecule present in a mixture, where the TAT fusion molecule comprises a TAT protein transduction domain covalently linked to a cargo moiety, comprising (a) contacting said mixture comprising the TAT fusion molecule with an immobilized binding protein of the invention under conditions such that the TAT fusion molecule binds to the immobilized binding protein; (b) eluting said TAT fusion molecule from the immobilized binding protein.

In some embodiments, the binding proteins of the invention can be used to enrich for intact TAT fusion molecules in a mixture. For example a mixture can comprise both intact and degraded forms of TAT fusion molecules.

In some embodiments, a binding protein of the invention is labeled with horseradish peroxidase, sulfotag, alkaline phosphatase, cresol violet, quantum dots, or fluorescein isothiocyanate (FITC), an infrared molecule, a radiolabel, or an EPR spin tracer label.

In one embodiment, an ELISA assay is used to detect and/or quantify a TAT peptide comprising a TAT protein transduction domain or a TAT fusion molecule comprising a TAT protein transduction domain. In an exemplary ELISA, binding proteins, e.g., antibodies binding to a TAT protein transduction domain, are immobilized onto a selected surface exhibiting protein affinity, such as a well in a polystyrene microtiter plate. Then, a test composition or sample, e.g., a blood or tissue sample containing a TAT protein transduction domain, e.g., a TAT protein transduction domain fused to a cargo moiety, is added to the wells. After binding and washing to remove non-specifically bound immunocomplexes, the bound antigen may be detected. Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing a TAT protein transduction domain, e.g., a TAT fusion molecule comprising a TAT protein transduction domain, are immobilized onto the well surface and then contacted with anti-TAT peptide antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating, incubating or binding, washing to remove non-specifically bound species, and detecting the bound immunecomplexes. These are described as follows.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

The phrase “under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25 to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple. Quantitation is then achieved by measuring the degree of color generation, e.g., using a visible spectra spectrophotometer.

In certain embodiments, an alternative approach for detection of TAT peptide using the anti-TAT peptide binding proteins of the invention is employing protein immunoprecipitation combined with mass spectrometry, e.g., tandem mass spectrometry, e.g., multiple reaction monitoring mass spectrometry (IP-MRM). IP-MRM is known in the art and is described, for example, in Lin et al. (Journal of Proteome Research, 2013, 12, 5996-6003).

B. Labeling

The invention provides a method for detecting a TAT peptide, e.g., a TAT protein transduction domain or a TAT fusion molecule comprising a TAT protein transduction domain, in a biological sample comprising contacting the biological sample with a binding protein, e.g., antibody, or antibody portion, of the invention and detecting the binding protein, e.g., antibody (or antibody portion) bound to the TAT peptide, e.g., a TAT protein transduction domain or a TAT fusion molecule comprising a TAT protein transduction domain, to thereby detect TAT peptide, e.g., a TAT protein transduction domain or a TAT fusion molecule comprising a TAT protein transduction domain, in the biological sample. The binding protein is directly or indirectly labeled with a detectable substance to facilitate detection of the bound or unbound antibody.

Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

In some embodiments, a binding protein of the invention is labeled with horseradish peroxidase, SULFO-TAG labeled Streptavidin, alkaline phosphatase, cresol violet, quantum dots, or fluorescein isothiocyanate (FITC), an infrared molecule, a radiolabel, or an electron paramagnetic resonance (EPR) spin tracer label.

One skilled in the art will recognize that many strategies can be used for labeling binding proteins of the invention to enable their detection or discrimination in a mixture of particles. The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner, e.g. a TAT peptide antibody as described herein, that binds to TAT peptide, e.g., a TAT protein transduction domain or a TAT fusion molecule comprising a TAT protein transduction domain, where the binding partner is attached to a fluorescent moiety. The compositions and methods of the invention may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. Moieties suitable for the compositions and methods of the invention are described in more detail below.

Fluorescent labels include near-infrared (NIR) fluorophores, which are described in, for example, Frangioni, 2003, Current Opinions in Chemical Biology, 7(5):626, the contents of which are hereby incorporated herein by reference.

In some embodiments, the invention provides a label for detecting a biological molecule comprising a binding partner for the biological molecule, e.g. a TAT peptide antibody as described herein, that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are Alexa Fluor molecules selected from the group consisting of Alexa Fluor 488, Alexa Fluor 532, Alexa Fluor 647, Alexa Fluor 680 or Alexa Fluor 700. In some embodiments, the dye molecules are Alexa Fluor molecules selected from the group consisting of Alexa Fluor 488, Alexa Fluor 532, Alexa Fluor 680 or Alexa Fluor 700. In some embodiments, the dye molecules are Alexa Fluor 647 dye molecules. In some embodiments, the dye molecules comprise a first type and a second type of dye molecules, e.g., two different Alexa Fluor molecules, e.g., where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4. The binding partner can be, e.g. a TAT peptide antibody as described herein.

In some embodiments, the invention provides a label for the detection of TAT peptide, e.g., a TAT protein transduction domain or a TAT fusion molecule comprising a TAT protein transduction domain, wherein the label comprises a binding partner for the TAT peptide and a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the fluorescent moiety comprises a fluorescent molecule. In some embodiments, the fluorescent moiety comprises a plurality of fluorescent molecules, e.g., about 2 to 10, 2 to 8, 2 to 6, 2 to 4, 3 to 10, 3 to 8, or 3 to 6 fluorescent molecules. In some embodiments, the label comprises about 2 to 4 fluorescent molecules. In some embodiments, the fluorescent dye molecules comprise at least one substituted indolium ring system in which the substituent on the 3-carbon of the indolium ring contains a chemically reactive group or a conjugated substance. In some embodiments, the fluorescent molecules are selected from the group consisting of Alexa Fluor 488, Alexa Fluor 532, Alexa Fluor 647, Alexa Fluor 680 or Alexa Fluor 700. In some embodiments, the fluorescent molecules are selected from the group consisting of Alexa Fluor 488, Alexa Fluor 532, Alexa Fluor 680 or Alexa Fluor 700. In some embodiments, the fluorescent molecules are Alexa Fluor 647 molecules. In some embodiments, the binding partner comprises an anti-TAT peptide antibody as described herein.

Electron paramagnetic resonance (EPR) spin tracer labelling can also be used to detect TAT peptide, e.g., a TAT protein transduction domain or a TAT fusion molecule comprising a TAT protein transduction domain, wherein the TAT antibodies are labeled with an EPR tracer (see, e.g., Hubbell et al., 1998, Current Opinion in Structural Biology, 8(5):649, the contents of which are hereby incorporated herein by reference).

Alternative to labeling the antibody, TAT peptide, e.g., a TAT protein transduction domain or a TAT fusion molecule comprising a TAT protein transduction domain, can be assayed in biological fluids by a competition immunoassay utilizing TAT peptide standards labeled with a detectable substance and an unlabeled TAT peptide antibody. In this assay, the biological sample, the labeled TAT peptide standards and the TAT peptide antibody are combined and the amount of labeled standard bound to the unlabeled antibody is determined. The amount of TAT peptide, e.g., a TAT protein transduction domain or a TAT fusion molecule comprising a TAT protein transduction domain, in the biological sample is inversely proportional to the amount of labeled standard bound to the anti-TAT peptide antibody. Similarly, TAT peptide, e.g., a TAT protein transduction domain or a TAT fusion molecule comprising a TAT protein transduction domain, can also be assayed in biological fluids by a competition immunoassay utilizing TAT peptide standards labeled with a detectable substance and an unlabeled TAT peptide antibody.

C. Therapeutic Uses of the Invention

The binding proteins, e.g., antibodies and antibody portions thereof, preferably are capable of neutralizing TAT activity both in vivo and in vitro.

It has previously been shown that TAT vaccination as well as the administration of antibodies against the TAT protein are protective against HIV-1 infection, and can result in long term suppression. In addition, in the case of acute exposure, infusion of anti-TAT antibodies could also block extracellular TAT autocrine/paracrine transactivation of HIV-1 replication (see, e.g., Moreau, et al. Journal of General Virology (2004), 85, 2893-2901; Bennasser, Y., et al. (2002). Virology 303, 174-180; Ensoli, B., et al. (1990). Nature, 345, 84-86; Moreau, E., et al. (2004). J Virol 78, 3792-3796; Re, M. C., Furlini, G., Vignoli, M. (1995). J Acquir Immune Defic Syndr Hum Retrovirol 10, 408-416; Re, M. C., Vignoli, et al. (2001). J Clin Virol 21, 81-89; Richardson, M. W., Mirchandani, J., et al. (2003). Biomed Pharmacother 57, 4-14; Tikhonov, I., et al. (2003) J Virol 77, 3157-3166; Steinaa, L., et al. (1994) Arch Virol 139, 263-271; Silvera, P., et al. (2002) J Virol 76, 3800-3809, the contents of which are incorporated herein by reference).

Accordingly, the binding proteins of the present invention can be used to inhibit TAT activity, e.g., in a cell culture, in human subjects or in other mammalian subjects, and thereby block or inhibit infection by HIV. In one embodiment, the disclosure provides a method for inhibiting TAT activity comprising contacting TAT with an binding protein, e.g., an antibody, or antigen-binding portion such that TAT activity is inhibited.

In another embodiment, disclosed herein is a method for reducing TAT activity in a subject, advantageously in a subject suffering from a TAT associated disease, e.g., HIV infection or AIDS. The disclosure provides methods for reducing TAT activity in a subject suffering from such a disease, which method comprises administering to the subject binding protein, e.g., an antibody or antibody portion of the disclosure such that TAT peptide activity in the subject is reduced. Preferably, the subject is a human subject.

In another embodiment, disclosed herein is a method for inhibiting the activity of HIV-TAT protein in a subject, comprising administering to the subject an antigen binding protein of the invention, thereby inhibiting activity of the HIV-TAT protein in the subject.

Binding proteins, e.g., antibodies of the disclosure can be administered to a human subject for therapeutic purposes. Moreover, binding proteins, e.g., antibodies of the disclosure can be administered to a non-human mammal comprising a TAT peptide with which the antibody is capable of binding for veterinary purposes or as an animal model of human disease. Regarding the latter, such animal models may be useful for evaluating the therapeutic efficacy of antibodies of the disclosure (e.g., testing of dosages and time courses of administration).

Non-limiting examples of diseases that can be treated with the binding proteins, e.g., antibodies, and antigen binding portions thereof, include HIV infection and AIDS, and associated symptoms thereof.

In another aspect, this application features a method of treating (e.g., curing, suppressing, ameliorating, inhibiting delaying or preventing the onset of, or preventing recurrence or relapse of) or preventing a TAT associated disorder, in a subject. The method includes: administering to the subject binding protein, e.g., an anti-TAT peptide antibody or portion thereof as described herein, in an amount sufficient to treat or prevent the TAT-associated disorder. The TAT antagonist, e.g., the anti-TAT antibody, or portion thereof, can be administered to the subject, alone or in combination with other therapeutic modalities as described herein.

Binding proteins, e.g., antibodies, or antigen binding portions thereof, can be used alone or in combination to treat such diseases. It should be understood that the antibodies or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody, e.g., HIV or AIDS, or associated symptoms thereof. The additional agent also can be an agent that imparts a beneficial attribute to the therapeutic composition, e.g., an agent which affects the viscosity of the composition.

It should further be understood that the combinations which are to be included within this disclosure are those combinations useful for their intended purpose. The agents set forth below are illustrative for purposes and not intended to be limited. The combinations, which are part of this disclosure, can be the antibodies of the disclosure and at least one additional agent. The combination can also include more than one additional agent, e.g., two or three additional agents if the combination is such that the formed composition can perform its intended function.

The combination therapy can include one or more TAT antagonists, e.g., anti-TAT antibodies, or portions thereof, formulated with, and/or co-administered with, one or more anti-HIV agent. The term “anti-HIV agent” refers to drugs used to inhibit or prevent HIV infection and AIDS, or to treat or ameliorate symptoms of HIV infection or AIDS. In one embodiment, the anti-TAT antibodies of the invention are administered in combination with one or more of an antiretroviral therapy (ART), including, but not limited to non-nucleoside reverse transcriptase inhibitors (NNRTIs) such as efavirenz (Sustiva™), etravirine (Intelence™) and nevirapine (Viramune™); nucleoside or nucleotide reverse transcriptase inhibitors (NRTIs) such as Abacavir™ (Ziagen™), and the combination drugs emtricitabine/tenofovir (Truvada™), Descovy™ (tenofovir alafenamide/emtricitabine), and lamivudine-zidovudine (Combivir™); protease inhibitors (PIs) such as atazanavir (Reyataz™), darunavir (Prezista™), fosamprenavir (Lexiva™) and indinavir (Crixivan™); entry or fusion inhibitors such as enfuvirtide (Fuzeon™) and maraviroc (Selzentry™); and integrase inhibitors such as raltegravir (Isentress™) and dolutegravir (Tivicay™).

In particular embodiments, the anti-TAT antibodies or ADCs can be administered alone or with another anti-HIV agent which acts in conjunction with or synergistically with the antibody to treat the disease associated with TAT activity.

Provided herein are methods for treating HIV or AIDS, in a patient comprising administering to the patient an anti-TAT binding protein, e.g., antibody, or fragment thereof, of the invention wherein the combination therapy exhibits synergy, e.g., therapeutic synergy, in the subject. As used herein, “synergy” or “therapeutic synergy” refers to a phenomenon where treatment of patients with a combination of therapeutic agents manifests a therapeutically superior outcome to the outcome achieved by each individual constituent of the combination used at its optimum dose. For example, a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components.

The pharmaceutical compositions may include a “therapeutically effective amount” or a “prophylactically effective amount” of an antibody or antibody portion. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

D. Diagnostic Uses of the Invention

In some embodiments, any of the anti-TAT antibodies provided herein is useful for detecting the presence of TAT and HIV in a biological sample. Detecting encompasses quantitative or qualitative detection.

The binding proteins, e.g., antibodies, disclosed herein can be used for a variety of purposes, such as for detecting an HIV infection or diagnosing AIDS in a subject. These methods can include contacting a sample from the subject diagnosed with HIV or AIDS with an binding protein, e.g., antibody, described herein, and detecting binding of the binding protein, e.g., antibody, to the sample. An increase in binding of the binding protein, e.g., antibody, to the sample relative to binding of the binding protein, e.g., antibody, to a control sample confirms that the subject has an HIV infection and/or AIDS. In some embodiments, the methods further comprise contacting a second antibody that binds TAT peptide with the sample, and detecting binding of the second antibody. In some non-limiting examples an increase in binding of the antibody to the sample relative to a control sample detects TAT peptide in the subject, and thus diagnoses HIV infection in the subject.

According to another embodiment, the present invention provides diagnostic methods. Diagnostic methods generally involve contacting a biological sample obtained from a patient, such as, for example, blood, serum, saliva, urine, sputum, a cell swab sample, or a tissue biopsy, with a TAT binding protein, e.g., antibody, and determining whether the binding protein, e.g., antibody, preferentially binds to the sample as compared to a control sample or predetermined cut-off value, thereby indicating the presence of a TAT peptide in the sample.

In some embodiments, an anti-TAT binding protein, e.g., antibody, for use in a method of diagnosis or detection is provided. In a further aspect, a method of detecting the presence of TAT peptide in a biological sample is provided. In some embodiments, the method comprises contacting the biological sample with an anti-TAT binding protein, e.g., antibody, as described herein under conditions permissive for binding of the anti-TAT binding protein, e.g., antibody, to a TAT peptide, and detecting whether a complex is formed between the anti-TAT binding protein, e.g., antibody, and a TAT peptide. Such method may be an in vitro or in vivo method. In some embodiments, an anti-TAT binding protein, e.g., antibody, is used to select subjects eligible for therapy with an anti-TAT binding protein, e.g., antibody, for example, where HIV infection is used for selection of patients.

Exemplary diseases that may be diagnosed using a binding protein, e.g., antibody, of the invention include TAT associated diseases such as diseases characterized by infection of HIV, including AIDS.

The methods of the present invention can be practiced in conjunction with any other method used by the skilled practitioner to provide a diagnosis of the occurrence or recurrence of a TAT associated disease. It is understood that the diagnostic and monitoring methods provided herein are typically used in conjunction with other methods known in the art. For example, the methods of the invention may be performed in conjunction with physical exam and other known methods for diagnosis.

Methods for assessing efficacy of a treatment regimen in a subject are also provided. In these methods the amount of TAT peptide in a pair of samples (a first sample obtained from the subject at an earlier time point or prior to the treatment regimen and a second sample obtained from the subject at a later time point, e.g., at a later time point when the subject has undergone at least a portion of the treatment regimen) is assessed. It is understood that the methods of the invention include obtaining and analyzing more than two samples (e.g., 3, 4, 5, 6, 7, 8, 9, or more samples) at regular or irregular intervals for assessment of TAT peptide levels. Pairwise comparisons can be made between consecutive or non-consecutive subject samples.

The invention provides methods for monitoring the treatment of a TAT associated disease in a subject by (1) contacting a first biological sample obtained from the subject prior to administering at least a portion of a treatment regimen to the subject with a detection reagent specific for a TAT peptide; (2) contacting a second biological sample obtained from the subject after administering at least a portion of a treatment regimen to the subject with a detection reagent specific for a TAT peptide; (3) measuring the amount of a TAT peptide detected in each the first biological sample and the second biological sample by each detection reagent; and (4) comparing the level of expression of the TAT peptide in the first sample with the level of the a TAT peptide in the second sample, thereby monitoring the treatment of the TAT associated disease in the subject.

In certain embodiments the diagnostic and monitoring methods provided herein further comprising obtaining a subject sample.

In certain embodiments the diagnostic and monitoring methods provided herein further comprising selecting a treatment regimen for the subject based on the level of TAT peptide detected.

1. Diagnostic Assays

The binding proteins, e.g., antibody, or antigen binding fragment thereof, of the invention are useful in methods of detecting, quantifying, isolating, and/or purifying a TAT peptide, e.g., a TAT protein transduction domain. In some embodiments of the methods provided herein, the TAT protein transduction domain is comprised in a TAT fusion molecule. Thus, in some embodiments, the binding proteins, e.g., antibody, or antigen binding fragment thereof, of the invention are useful in methods of detecting, quantifying, isolating, and/or purifying a TAT fusion molecule. It will be understood that any of the methods provided herein can be applied to a TAT peptide, a TAT protein transduction domain, and/or to a TAT fusion molecule.

An exemplary method for detecting the presence or absence or amount or level of a TAT peptide, e.g., a TAT protein transduction domain, or a TAT fusion molecule, in a biological sample involves obtaining a biological sample from a test subject and contacting the biological sample with a binding protein, e.g., antibody, or antigen binding fragment thereof, of the invention to detect the TAT peptide, e.g., a TAT protein transduction domain, or a TAT fusion molecule.

Methods provided herein for detecting the presence or absence or amount or level of a TAT peptide, e.g., a TAT protein transduction domain, or a TAT fusion molecule in a biological sample include obtaining a biological sample from a subject that may or may not contain the TAT peptide, e.g., a TAT protein transduction domain, or a TAT fusion molecule to be detected, contacting the sample with a TAT peptide binding protein, e.g., antibody, or antigen binding fragment thereof, as described herein, and contacting the sample with a detection reagent for detection of the TAT peptide, e.g., TAT protein transduction domain-specific binding agent complex, or a TAT fusion molecule-specific binding agent complex, if formed.

The methods include formation of either a transient or stable complex between the TAT peptide, e.g., TAT protein transduction domain, or a TAT fusion molecule, and the TAT peptide antibody, or antigen binding fragment thereof as described herein. The methods require that the complex, if formed, be formed for sufficient time to allow a detection reagent to bind the complex and produce a detectable signal (e.g., fluorescent signal, a signal from a product of an enzymatic reaction, e.g., a peroxidase reaction, a phosphatase reaction, a beta-galactosidase reaction, or a polymerase reaction).

An intact antibody, or a fragment or derivative thereof (e.g., Fab or F(ab′)₂) can be used in the methods of the invention. Such strategies of TAT peptide detection are used, for example, in ELISA, RIA, immunoprecipitation, western blot, antibody-labeled fluorescence imaging, tissue immunohistochemistry, immunoprecipitation or immunopurification followed by mass spectrometry, e.g., Immunoprecipitation-Multiple Reaction Monitoring (IPMRM), and immunofluorescence assay methods. In certain embodiments, e.g., in an ELISA, RIA, immunoprecipitation assay, western blot, immunofluorescence assay, the TAT peptide-specific binding agent complex is attached to a solid support for detection of the TAT peptide, e.g., TAT protein transduction domain, or a TAT fusion molecule. The complex can be formed on the substrate or formed prior to capture on the substrate. For in-gel enzyme assays, the TAT peptide, e.g., TAT protein transduction domain, or a TAT fusion molecule, is resolved in a gel, typically an acrylamide gel, in which a substrate for the enzyme is integrated.

In yet another aspect, this application provides a method for detecting the presence of TAT peptide, e.g., TAT protein transduction domain, or a TAT fusion molecule, in vivo (e.g., in vivo imaging in a subject). The subject method can be used to detect the presence of the TAT peptide, e.g., TAT protein transduction domain, or to detect or quantify a TAT fusion molecule, or to determine localization or verify delivery of a TAT fusion molecule in a subject. In exemplary embodiments, the method includes: (i) administering the anti-TAT peptide antibody or fragment thereof as described herein to a subject or a control subject under conditions that allow binding of the antibody or fragment to TAT peptide; and (ii) detecting formation of a complex between the antibody or fragment and TAT peptide, wherein a statistically significant change in the formation of the complex in the subject relative to the control subject is indicative of the presence of TAT peptide, e.g., TAT protein transduction domain, or a TAT fusion molecule.

In some embodiments, methods provided herein comprise detecting the presence or absence or amount or level of a TAT peptide, e.g., a TAT protein transduction domain, or a TAT fusion molecule, in a biological sample, by contacting a biological sample obtained from a subject with a TAT peptide binding protein, e.g., antibody, or antigen binding fragment thereof, of the invention to form a complex between the TAT peptide, e.g., TAG protein transduction domain, or the TAT fusion molecule, and the TAT peptide binding protein, and purifying said complex. In some embodiments, the TAT peptide binding protein may be immobilized on a solid support, e.g., a plate, a bead, or a chromatography resin. In further embodiments, the methods provided herein may comprise a mass spectrometry-based analysis to detect the presence or absence or amount or level of the TAT peptide, e.g., a TAT protein transduction domain, or a TAT fusion molecule after it has been purified as a part of the complex as described above.

In some embodiments, methods provided herein comprise detecting the presence or absence or amount or level of a TAT fusion molecule by contacting a biological sample obtained from a subject with a TAT-peptide binding protein, e.g., antibody, or antigen binding fragment thereof, of the invention to form a complex between the TAT fusion molecule and the TAT-peptide binding protein; purifying said complex; and analyzing at least a portion of the TAT fusion molecule by mass spectrometry. In some embodiments, the TAT peptide binding protein may be immobilized on a solid support, e.g., a plate, a bead, or a chromatography resin.

In some embodiments, methods provided herein comprise detecting the presence or absence or amount or level of a TAT fusion molecule by contacting a biological sample obtained from a subject with a TAT-peptide binding protein, e.g., antibody, or antigen binding fragment thereof, of the invention immobilized on a solid support to form a complex between the TAT fusion molecule and the TAT-peptide binding protein; purifying said complex; treating said complex with a protease, e.g., trypsin, to generate at least one peptide derived from a TAT fusion molecule; and analyzing said at least one peptide by mass spectrometry. In some embodiments, the TAT fusion molecule may be a TAT-frataxin fusion molecule.

It was found that TAT-peptide binding proteins known in the art, such as the commercially available anti-TAT mouse monoclonal IgM antibody from Abcam (ab63957), when used in a method for detecting the presence or absence or amount or level of a TAT-frataxin fusion molecule in a biological sample, were characterized by inadequate performance. For example, it was found that, when a commercially available anti-TAT antibody was used for immunopurification of a TAT-frataxin fusion molecule from a biological sample, followed by digesting the immunopurified complex with trypsin and measuring levels of the tryptic peptides by liquid chromatography and mass spectrometry (LC/MS), the resulting mass spectrometric signal was lower than the mass spectrometric signal obtained in a similar experiment in which a commercially available anti-frataxin antibody was used for immunopurification. This result indicated that commercially available anti-TAT antibody was not capable of immunopurifying sufficient amounts of TAT-frataxin fusion molecule from a biological sample to allow reliable detection and quantification of the TAT-frataxin fusion molecule. The TAT-peptide binding proteins of the invention overcome the problem of inadequate performance described above and allow reliable detection and quantification of a TAT fusion molecule, e.g., a TAT-frataxin fusion molecule.

Accordingly, in some aspects, provided are methods for detecting the presence of a TAT fusion molecule, or quantifying the level of a TAT fusion molecule in a sample, comprising contacting the sample with a TAT peptide binding protein, e.g., antibody, or antigen binding fragment thereof, of the invention to form a complex between the TAT fusion molecule and the TAT-peptide binding protein, thereby detecting the presence of a TAT fusion molecule, or quantifying the level of a TAT fusion molecule, in the sample,

wherein the TAT peptide binding protein exhibits a specific binding affinity to the TAT fusion molecule that is higher than the specific binding affinity to the TAT fusion molecule of anti-TAT mouse monoclonal IgM antibody from Abcam (ab63957). In some embodiments, the specific binding affinity is measured at the conditions that comprise one or more of ambient temperature (e.g., 20-25° C.); pH of about 7.4; and phosphate-buffered saline (PBS) buffer.

In some embodiments, the TAT peptide binding protein of the invention may be characterized by a K_{D} that is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 100-fold or at least about 500-fold lower (i.e., greater affinity) than K_{D} of ab63957. In some embodiments, the K_{D} is measured at the conditions that comprise one or more of ambient temperature (e.g., 20-25° C.); pH of about 7.4; and phosphate-buffered saline (PBS) buffer.

In some embodiments, the TAT fusion molecule is TAT frataxin fusion molecule. In some embodiments, the sample may be a biological sample, e.g., a liquid sample such as a blood sample, a plasma sample or a serum sample, or a solid sample (e.g., a tissue sample, such as a skin sample or a buccal sample).

In some aspects, provided are methods for isolating or purifying a TAT fusion molecule from a sample, comprising contacting the sample with a TAT peptide binding protein, e.g., antibody, or antigen binding fragment thereof, of the invention to form a complex between the TAT fusion molecule and the TAT-peptide binding protein; and isolating or purifying said complex from the sample;

wherein the TAT peptide binding protein exhibits a specific binding affinity to the TAT fusion molecule that is higher than the specific binding affinity to the TAT fusion molecule of anti-TAT mouse monoclonal IgM antibody from Abcam (ab63957). In some embodiments the specific binding is measured at the conditions that comprise one or more of ambient temperature (e.g., 20-25° C.); pH of about 7.4; and phosphate-buffered saline (PBS) buffer.

In some embodiments, the TAT peptide binding protein of the invention may be characterized by a K_{D} that is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 100-fold or at least about 500-fold lower (i.e., greater affinity) than K_{D} of ab63957. In some embodiments, the K_{D} is measured at the conditions that comprise one or more of ambient temperature (e.g., 20-25° C.); pH of about 7.4; and phosphate-buffered saline (PBS) buffer.

In some embodiments, the TAT fusion molecule is TAT frataxin fusion molecule. In some embodiments, the sample may be a biological sample, e.g., a liquid sample such as a blood sample, a plasma sample or a serum sample, or a solid sample (e.g., a tissue sample, such as a skin sample or a buccal sample).

In some aspects, provided are methods for detecting the presence of a TAT fusion molecule, or quantifying the level of a TAT fusion molecule in a sample, comprising:

(a) contacting the sample with a TAT peptide binding protein, e.g., antibody, or antigen binding fragment thereof, of the invention to form a complex between the TAT fusion molecule and the TAT-peptide binding protein,

(b) purifying said complex;

(c) treating said complex with a protease, e.g., trypsin, to generate at least one peptide derived from the TAT fusion molecule; and

(d) analyzing said at least one peptide by mass spectrometry, thereby generating a mass spectrometric signal corresponding to said peptide;

wherein said mass spectrometric signal generated in step (d) is at least about 2 to about 10-fold higher, e.g., at least about 3-fold higher, at least about 4-fold higher, at least about 5-fold higher, at least about 6-fold higher, at least about 7-fold higher, at least about 8-fold higher, at least about 9-fold higher or at least about 10-fold higher than the mass spectrometric signal generated when step (a) is carried out using the anti-TAT mouse monoclonal IgM antibody from Abcam (ab63957).

In some embodiments, step (a) may be carried out at the conditions that comprise one or more of ambient temperature (e.g., 20-25° C.); pH of about 7.4; and phosphate-buffered saline (PBS) buffer.

In some embodiments, the TAT fusion molecule is TAT frataxin fusion molecule. In some embodiments, the sample may be a biological sample that may be a liquid sample (e.g., a blood sample, a plasma sample or a serum sample) or a solid sample (e.g., a tissue sample, such as a skin sample or a buccal sample).

E. Kits

The present invention provides compositions and kits for the detection, quantification, purification and/or isolation of TAT peptide, and in particular a TAT fusion molecule comprising a TAT protein transduction domain. The invention also provides compositions and kits for diagnosing or monitoring a TAT associated disease or recurrence of a TAT associated disease.

These kits include one or more of the following: a detectable binding protein, e.g., antibody, that specifically binds to a TAT peptide, e.g., a TAT protein transduction domain or a TAT fusion molecule comprising a TAT protein transduction domain, reagents for obtaining and/or preparing subject tissue samples for staining, and instructions for use. In one embodiment, the binding protein, e.g., antibody, is any one or more of the binding proteins described herein.

The invention also encompasses kits for detecting the presence of a TAT peptide, e.g., a TAT protein transduction domain or a TAT fusion molecule comprising a TAT protein transduction domain, in a biological sample. Such kits can be used to detect, quantify, purify and/or isolate a TAT protein transduction domain or a TAT fusion molecule comprising a TAT protein transduction domain. Such kits can also be used to determine if a subject is suffering from a TAT associated disease. For example, the kit can comprise a labeled compound or agent capable of detecting a TAT peptide, e.g., a TAT protein transduction domain or a TAT fusion molecule comprising a TAT protein transduction domain, in a biological sample and means for detecting and/or quantifying and/or isolating and/or purifying the TAT peptide, e.g., a TAT protein transduction domain or a TAT fusion molecule comprising a TAT protein transduction domain, in the sample.

Kits can also include instructions for use of the kit for practicing any of the methods provided herein or interpreting the results obtained using the kit based on the teachings provided herein. The kits can also include reagents for detection of a control protein in the sample not related to the TAT associated disease, e.g., actin for tissue samples, albumin in blood or blood derived samples for normalization of the amount of the target antigen present in the sample. The kit can also include the purified TAT peptide, e.g., a TAT fusion molecule comprising a TAT protein transduction domain, for detection for use as a control or for quantitation of the assay performed with the kit.

Kits include reagents for use in a method to diagnose a TAT associated disease, e.g., HIV invention or AIDS, in a subject, the kit comprising a detection reagent, e.g. an antibody of the invention, wherein the detection reagent is specific for a TAT peptide, e.g., a TAT protein transduction domain or a TAT fusion molecule comprising a TAT protein transduction domain. In one embodiment, the detection reagent is any one or more of the binding proteins described herein.

For antibody-based kits, the kit can comprise, for example: (1) a first antibody (e.g., attached to a solid support) which binds to a first target protein (e.g., TAT peptide); and, optionally, (2) a second, different antibody which binds to either the first target protein or the first antibody and is conjugated to a detectable label.

Reagents specific for detection of a TAT peptide, e.g., a TAT fusion molecule comprising a TAT protein transduction domain, allow for detection and quantitation of the a TAT peptide, e.g., a TAT fusion molecule comprising a TAT protein transduction domain, in a complex mixture, e.g., serum or tissue sample. In certain embodiments, the kits for the detection, quantification, isolation or purification of TAT peptide, e.g., a TAT fusion molecule comprising a TAT protein transduction domain, or for the diagnosis or monitoring of a TAT associated disease comprise at least one reagent specific for the detection of a TAT peptide, e.g., a TAT fusion molecule comprising a TAT protein transduction domain.

In certain embodiments, the kits further comprise instructions for the detection, quantification, isolation or purification of TAT peptide, e.g., a TAT fusion molecule comprising a TAT protein transduction domain, or for the diagnosis or monitoring of a TAT associated disease based on the level of expression of the TAT peptide, e.g., a TAT fusion molecule comprising a TAT protein transduction domain.

In certain embodiments, the kits can also comprise any one of, but not limited to, a buffering agent(s), a preservative, a protein stabilizing agent, reaction buffers. The kit can further comprise components necessary for detecting the detectable label (e.g., an enzyme or a substrate). The kit can also contain a control sample or a series of control samples which can be assayed and compared to the test sample. The controls can be control serum samples or control samples of purified proteins or nucleic acids, as appropriate, with known levels of target TAT peptide. Each component of the kit can be enclosed within an individual container and all of the various containers can be within a single package, along with instructions for interpreting the results of the assays performed using the kit.

The kits of the invention may optionally comprise additional components useful for performing the methods of the invention.

### IV. Pharmaceutical Compositions

The invention also provides pharmaceutical compositions comprising a binding protein, e.g., antibody, or antigen-binding portion thereof, of the invention and a pharmaceutically acceptable carrier. The pharmaceutical compositions comprising antibodies of the invention are for use in, but not limited to, treatment of a TAT associated disease, and/or in research. In a specific embodiment, a pharmaceutical composition comprises one or more binding proteins, e.g., antibodies of the invention. In accordance with these embodiments, the composition may further comprise of a carrier, diluent or excipient.

The binding proteins, e.g., antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

Various delivery systems are known and can be used to administer one or more antibodies of the invention, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering an antibody of the invention include, but are not limited to, parenteral administration (e.g., intradermal, intramuscular, intraperitoneal, intravenous and subcutaneous), epidural administration, intratumoral administration, and mucosal administration (e.g., intranasal and oral routes).

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

As will be appreciated by the skilled artisan, the route and/or mode of administration will vary depending upon the desired results.

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### EXAMPLES

### Example 1: Generating Anti-TAT Peptide Antibodies

Experiments were performed to generate rabbit monoclonal antibodies against the TAT protein transduction domain. Polyclonal rabbit and monoclonal antibodies were produced, as well as expression plasmids encoding rabbit immunoglobulins for recombinant production of monoclonal antibodies against a TAT-moiety, the TAT protein transduction domain.

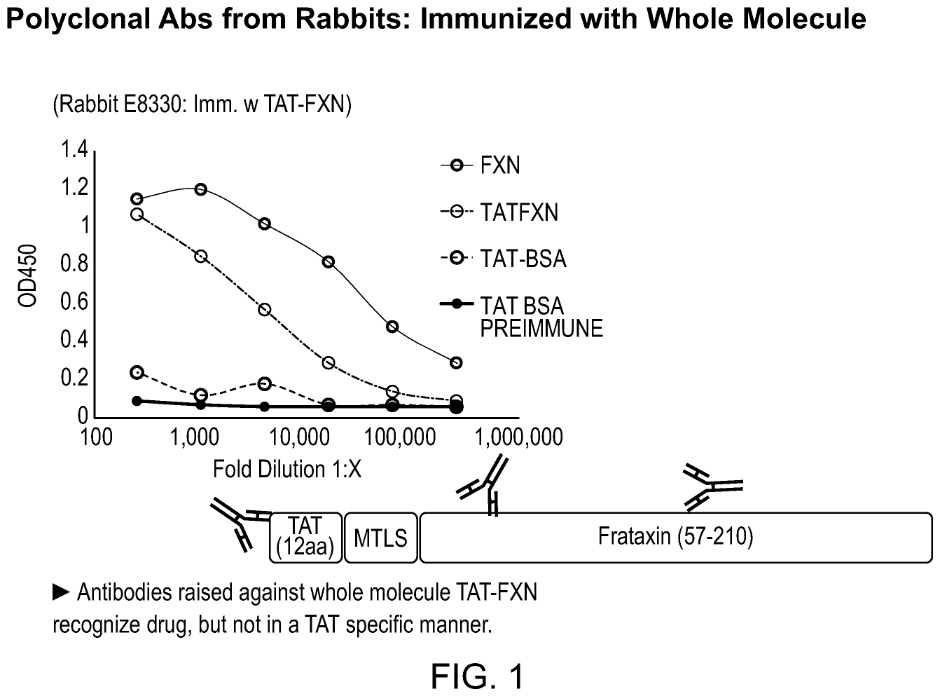

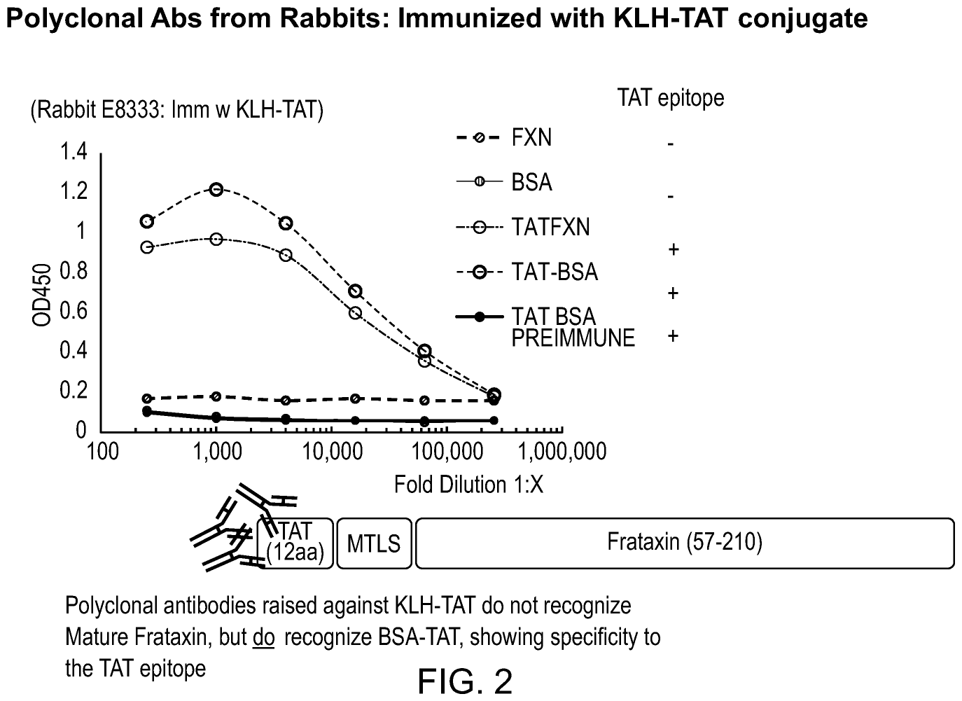

It was found that polyclonal antibodies raised against the entire TAT-FXN fusion molecule recognize drug, but not in a TAT specific manner (see FIG. 1). However, polyclonal antibodies raised against KLH-TAT do not recognize mature frataxin, but do recognize BSA-TAT, showing specificity to the TAT epitope (see FIG. 2).

Briefly, rabbits were immunized with TAT, followed by serum collection, affinity purification, and splenic hybridoma fusion to create rabbit monoclonal antibodies (RabMab).

Lead optimization included post-fusion hybridoma selection, RabMab expression characterization via EIA screening, limiting dilution plating, and single cell clone isolation and VH and VL sequencing followed by multi-milligram production of antibodies.

Methods

Immunizations in Rabbits

Polyclonal antibodies were obtained by immunizing rabbit E8332 with a TAT peptide antigen (MYGRKKRRQRRR-C, SEQ ID NO: 46) with a KLH or OVA conjugation. The TAT peptide antigen utilized comprises the sequence of the TAT protein transduction domain YGRKKRRQRRR (SEQ ID NO: 23). Rabbits received 4 rounds of TAT peptide by subcutaneous injection before a first bleed (bleed 1), followed by one additional injection and a second bleed (bleed 2). Following bleed 2, TAT peptide was iv injected, followed by isolation of spleen cells. Bleeds 1 and 2 were pooled and purified by affinity purification with TAT peptide antigen to generate polyclonal anti-TAT E8332 polyclonal antibody. Sera titer data and ELISA showed that E8332 was reactive to both TAT frataxin fusion molecule and TAT peptide antigen but had no reactivity to frataxin alone.

Hybridoma technology was used to generate anti TAT monoclonal antibodies. Following fusion of spleen cells and myeloma cells, anti TAT hybridomas were selected, and colonies were grown in semi-sold HAT selection medium (hypoxanthine-aminopterin-thymidine medium). Colonies were grown to sufficient numbers in 24 well multi-cell cultures. Single cell seeding was carried out in 40 96-well plates, such that there was isolation of colonies from a single hybridoma cell per well. Lastly, ELISA was carried out against platebound BSA-TAT-frataxin fusion protein and BSA-frataxin. Multiclone screening against dual antigens (subtractive interpretation) allowed for identification of clones against TAT in the form of a TAT frataxin fusion molecule (BSA-TAT-frataxin), but not against frataxin alone (BSA-frataxin).

Cloning VH and VL Sequences from Hybridomas

For determination of CDR sequences, total RNA was isolated from hybridoma cells. First and second-strand cDNA synthesis was performed. PCR products were separated by agarose electrophoresis and fragments were excised and purified. Fragments were cloned directly into expression vectors. Colonies from each reaction were scaled up for miniprep-scale plasmid purification.

Identification of Functional, Recombinant VH and VL Sequences

For each hybridoma, each plasmid was sequenced. These plasmids were subjected to DNA sequence determination and analysis.

Results

Polyclonal Antibodies

An experiment was conducted to compare the ability of a rabbit polyclonal antibody generated as described in the methods section herein and commercially available anti-frataxin (ab124680, ab113691, ab110328) and anti-TAT (ab63957) antibodies to immunopurify an exemplary TAT frataxin fusion molecule. The antibodies used in the experiment are described in the table below. The commercially available antibodies were from Abcam.

| Antibody Specificity | Antibody Description | Abcam Number |
|---|---|---|
| Anti-Frataxin | Rabbit monoclonal IgG | ab124680 |
| Anti-Frataxin | Mouse monoclonal | ab113691 |
| | IgG1 | |
| Anti-Frataxin | Mouse monoclonal | ab110328 |
| | IgG1 | |
| Anti-TAT | Mouse monoclonal IgM | ab63957 |
| Anti-TAT | Rabbit polyclonal | N/A |
| | | Generated as described in |
| | | the methods section herein |

The five antibodies were first biotinylated and then conjugated to streptavidin coated magnetic beads. Antibody-conjugated beads were added to the plasma samples containing an exemplary TAT frataxin fusion molecule and incubated in PBS at room temperature for the binding to occur. Following incubation, beads were washed with PBS and released in digestion buffer. Trypsin was added to generate tryptic peptides of the exemplary TAT frataxin fusion molecule. Following digestion, formic acid was added to stop the digestion, beads were removed and the digested samples were transferred into a clean plate for injection on the liquid chromatography/mass spectrometry (LC/MS) system. In the LC/MS experiment, peak intensitives corresponding to frataxin-derived tryptic peptide GLNQIWNVK (SEQ ID NO: 47) were determined.

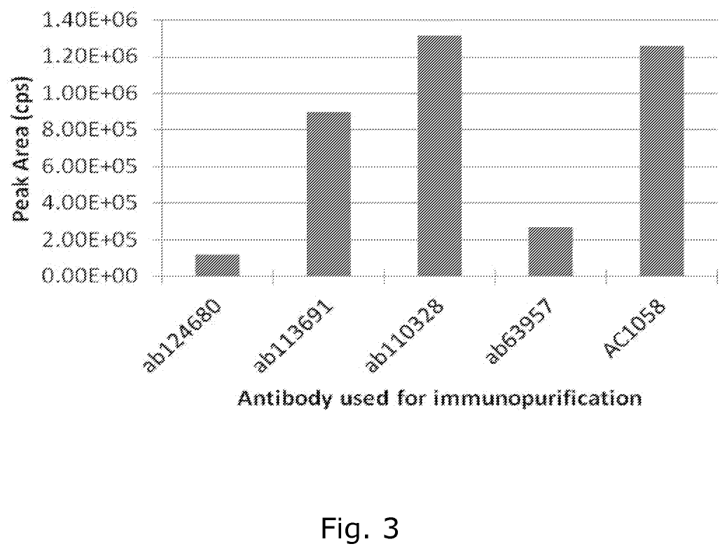

The results of the experiment are presented in FIG. 3. Specifically, FIG. 3 is a bar graph showing relative peak area generated by frataxin-derived tryptic peptide GLNQIWNVK (SEQ ID NO: 47) after immunopurification of an exemplary TAT frataxin fusion molecule using the tested antibodies. The results presented in FIG. 3 indicate that the LC/MS signal generated when anti-TAT antibody ab63957 is used for immunopurification is about 5-fold lower than the LC/MS signal generated when the anti-Frataxin antibody ab110328 is used. The results also indicate that the use of the anti-TAT polyclonal rabbit antibody for immunopurification results in an LC/MS signal that is about 5-fold higher than when the commercially available anti-TAT antibody ab63957 is used, and further, is similar to the LC/MS signal generated when the anti-Frataxin antibody ab110328 is used. These results demonstrate that the polyclonal rabbit antibody generated is much more efficient in immunopurifying an exemplary TAT frataxin fusion molecule than the commercially available anti-TAT antibody ab63957. These results indicate that the subsequently generated anti-TAT monoclonal antibodies will have at least the same or superior ability to immunopurify a TAT fusion molecule as the polyclonal antibody.

Monoclonal Antibodies

Monoclonal antibodies against the TAT protein transduction domain were generated by hybridoma procedures as described herein.

10 antibodies were converted to recombinant antibodies against the TAT protein transduction domain. The antibodies are referred to as 10-1, 10-4, 10-5, 10-9, 10-12, 12-1, 12-3, 12-8, 12-10, and 6.3. Complete amino acid sequences of the heavy and light chains from these 10 antibodies are set forth in Table 1, below, and as SEQ ID NOs: 1-22.

Antibodies 10-4, 10-5, 12-1, 12-3 have the same heavy and light chain variable region sequences; antibodies 10-9, 12-8, 12-10 have the same heavy and light chain variable region sequences; antibodies 10-12 and antibody 10-1 have the same heavy and light chain variable region sequences. Antibody 6.3 has unique heavy and light chain variable region sequences.

All but one antibody (antibody 6.3) share the same heavy chain CDR sequences. Thus, there is one set of consensus heavy chain CDRs for high affinity anti-TAT monoclonal antibodies (SEQ ID NOs: 2, 3, and 4).

Antibodies 10-1, 10-9, 10-12, 12-8 and 12-10 share the same light chain CDR sequences, and antibodies 10-4, 10-5, 12-1 and 12-3 share the same light chain CDR sequences. Thus, there are two sets of consensus light chain CDRs for high affinity anti-TAT monoclonal antibodies (SEQ ID NOs: 6, 7, and 8 and SEQ ID NOs: 10, 11, and 12).

TABLE 1
Anti-TAT Human Antibody Heavy And Light Chain Variable Region
Amino Acid Sequences

| SEQ ID | Antibody | Protein | |
|---|---|---|---|
| NO: | Name | Domain | Amino Acid Sequence |
| 1 | 10-1 | VH | QSLEESGGDLVKPGASLTLTCTASGFSFSSGYWI |
| | | | C |
| | | | WVRQAPGKGLEWIACIYTGDGDTSYASWAKGR |
| | | | FTISKTSSTTVTLQMTSLTVADTAIYFCARDTSGT |
| | | | FYYNLWGPGTLVAVSS |
| 2 | 10-1 | CDR-H1 | FSSGYWIC |
| 3 | 10-1 | CDR-H2 | CIYTGDGDTSYASWAKG |
| 4 | 10-1 | CDR-H3 | DTSGTFYYNL |
| 5 | 10-1 | VL | ELVLTQTASSVSAAVGGTVTISCQSSESVYKTNY |
| | | | LSWFQKKPGQPPKLLIYDASTLASGVPSRFSGSG |
| | | | SGTQFTLTISDLECDDAATYYCAGGYSDDINAFG |
| | | | GGTEVVVK |
| 6 | 10-1 | CDR-L1 | QSSESVYKTNYLS |
| 7 | 10-1 | CDR-L2 | DASTLAS |
| 8 | 10-1 | CDR-L3 | AGGYSDDINA |
| 1 | 10-4 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 10-4 | CDR-H1 | FSSGYWIC |
| 3 | 10-4 | CDR-H2 | CIYTGDGDTSYASWAKG |
| 4 | 10-4 | CDR-H3 | DTSGTFYYNL |
| 9 | 10-4 | VL | DVVMTQTPSPVSAPVGGTVTINCQASQNIYSNL |
| | | | AWYQQKPGQPPKLLIYGASTLASGVSSRFKGSR |
| | | | SGTEFTLTISDLECADAATYYCQSYVYSSSTADT |
| | | | FGGGTKVVVE |
| 10 | 10-4 | CDR-L1 | QASQNIYSNLA |
| 11 | 10-4 | CDR-L2 | GASTLAS |
| 12 | 10-4 | CDR-L3 | QSYVYSSSTADT |
| 1 | 10-5 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 10-5 | CDR-H1 | FSSGYWIC |
| 3 | 10-5 | CDR-H2 | CIYTGDGDTSYASWAKG |
| 4 | 10-5 | CDR-H3 | DTSGTFYYNL |
| 9 | 10-5 | VL | DVVMTQTPSPVSAPVGGTVTINCQASQNIYSNL |
| | | | AWYQQKPGQPPKLLIYGASTLASGVSSRFKGSR |
| | | | SGTEFTLTTSDLECADAATYYCQSYVYSSSTADT |
| | | | FGGGTKVVVE |
| 10 | 10-5 | CDR-L1 | QASQNIYSNLA |
| 11 | 10-5 | CDR-L2 | GASTLAS |
| 12 | 10-5 | CDR-L3 | QSYVYSSSTADT |
| 1 | 10-9 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 10-9 | CDR-H1 | FSSGYWIC |
| 3 | 10-9 | CDR-H2 | CIYTGDGDTSYASWAKG |
| 4 | 10-9 | CDR-H3 | DTSGTFYYNL |
| 13 | 10-9 | VL | AAVLTQTPSSVSAAVGGTVTISCQSSESVYKTNY |
| | | | LSWFQKKPGQPPKLLIYDASTLASGVPSRFSGSG |
| | | | SGTQFTLTISDLECDDAATYYCAGGYSDDINAFG |
| | | | GGTEVEIK |
| 6 | 10-9 | CDR-L1 | QSSESVYKTNYLS |
| 7 | 10-9 | CDR-L2 | DASTLAS |
| 8 | 10-9 | CDR-L3 | AGGYSDDINA |
| 14 | 10-12 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 10-12 | CDR-H1 | FSSGYWIC |
| 3 | 10-12 | CDR-H2 | CIYTGDGDTSYASWAKG |
| 4 | 10-12 | CDR-H3 | DTSGTFYYNL |
| 5 | 10-12 | VL | ELVLTQTASSVSAAVGGTVTISCQSSESVYKTNY |
| | | | LSWFQKKPGQPPKLLIYDASTLASGVPSRFSGSG |
| | | | SGTQFTLTISDLECDDAATYYCAGGYSDDINAFG |
| | | | GGTEVVVK |
| 6 | 10-12 | CDR-L1 | QSSESVYKTNYLS |
| 7 | 10-12 | CDR-L2 | DASTLAS |
| 8 | 10-12 | CDR-L3 | AGGYSDDINA |
| 1 | 12-1 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 12-1 | CDR-H1 | FSSGYWIC |
| 3 | 12-1 | CDR-H2 | CIYTGDGDTSYASWAKG |
| 4 | 12-1 | CDR-H3 | DTSGTFYYNL |
| 9 | 12-1 | VL | DVVMTQTPSPVSAPVGGTVTINCQASQNIYSNL |
| | | | AWYQQKPGQPPKLLIYGASTLASGVSSRFKGSR |
| | | | SGTEFTLTISDLECADAATYYCQSYVYSSSTADT |
| | | | FGGGTKVVVE |
| 10 | 12-1 | CDR-L1 | QASQNIYSNLA |
| 11 | 12-1 | CDR-L2 | GASTLAS |
| 12 | 12-1 | CDR-L3 | QSYVYSSSTADT |
| 1 | 12-3 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 12-3 | CDR-H1 | FSSGYWIC |
| 3 | 12-3 | CDR-H2 | CIYTGDGDTSYASWAKG |
| 4 | 12-3 | CDR-H3 | DTSGTFYYNL |
| 9 | 12-3 | VL | DVVMTQTPSPVSAPVGGTVTINCQASQNIYSNL |
| | | | AWYQQKPGQPPKLLIYGASTLASGVSSRFKGSR |
| | | | SGTEFTLTISDLECADAATYYCQSYVYSSSTADT |
| | | | FGGGTKVVVE |
| 10 | 12-3 | CDR-L1 | QASQNIYSNLA |
| 11 | 12-3 | CDR-L2 | GASTLAS |
| 12 | 12-3 | CDR-L3 | QSYVYSSSTADT |
| 1 | 12-8 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 12-8 | CDR-H1 | FSSGYWIC |
| 3 | 12-8 | CDR-H2 | CIYTGDGDTSYASWAKG |
| 4 | 12-8 | CDR-H3 | DTSGTFYYNL |
| 13 | 12-8 | VL | AAVLTQTPSSVSAAVGGTVTISCQSSESVYKTNY |
| | | | LSWFQKKPGQPPKLLIYDASTLASGVPSRFSGSG |
| | | | SGTQFTLTISDLECDDAATYYCAGGYSDDINAFG |
| | | | GGTEVEIK |
| 6 | 12-8 | CDR-L1 | QSSESVYKTNYLS |
| 7 | 12-8 | CDR-L2 | DASTLAS |
| 8 | 12-8 | CDR-L3 | AGGYSDDINA |
| 1 | 12-10 | VH | QSLEESGGDLVKPGASLTLTCTASGFSFSSGYWI |
| | | | CWVRQAPGKGLEWIACIYTGDGDTSYASWAKG |
| | | | RFTISKTSSTTVTLQMTSLTVADTAIYFCARDTS |
| | | | GTFYYNLWGPGTLVAVSS |
| 2 | 12-10 | CDR-H1 | FSSGYWIC |
| 3 | 12-10 | CDR-H2 | CIYTGDGDTSYASWAKG |
| 4 | 12-10 | CDR-H3 | DTSGTFYYNL |
| 13 | 12-10 | VL | AAVLTQTPSSVSAAVGGTVTISCQSSESVYKTNY |
| | | | LSWFQKKPGQPPKLLIYDASTLASGVPSRFSGSG |
| | | | SGTQFTLTISDLECDDAATYYCAGGYSDDINAFG |
| | | | GGTEVEIK |
| 6 | 12-10 | CDR-L1 | QSSESVYKTNYLS |
| 7 | 12-10 | CDR-L2 | DASTLAS |
| 8 | 12-10 | CDR-L3 | AGGYSDDINA |
| 15 | 6.3 | VH | QSLEESGGGLVKPEGSLTLTCTASGFSFGSGSWI |
| | | | CWVRQAPGKGLEWIACIYGSGSGDTAYATWAK |
| | | | GRFTISKTSSTTVTLQMTSLTAADTATYFCARDS |
| | | | DYVDFDLWGPGTLVTVSS |
| 16 | 6.3 | CDR-H1 | FGSGSWIC |
| 17 | 6.3 | CDR-H2 | CIYGSGSGDTAYATWAKG |
| 18 | 6.3 | CDR-H3 | DSDYVDFDL |
| 19 | 6.3 | VL | AQVLTQTPSSTSAAVGGTVTINCQSSQSVYKNN |
| | | | YLS |
| | | | WFQQKPGQPPKLLIYLASTLASGVPSRFSGSGSG |
| | | | TQFTLTISDLECDDAATYYCAGGYSDDICTFGGG |
| | | | TEVVVK |
| 20 | 6.3 | CDR-L1 | QSSQSVYKNNYLS |
| 21 | 6.3 | CDR-L2 | LASTLAS |
| 22 | 6.3 | CDR-L3 | AGGYSDDICT |

The nucleic acid sequences of the heavy and light chain variable regions of the 10 antibodies are set forth in Table 2, below.

TABLE 2
Anti-TAT Human Antibody Heavy And Light Chain Variable Region
Nucleic Acid Sequences

| SEQ ID | Antibody | | | |
|---|---|---|---|---|
| NO: | Name | Domain | Nucleic Acid Sequence | |
| 25 | 10-1 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 26 | 10-1 | VL | 1 | gagcttgtgc tgacccagac tgcatcgtcc gtgtctgcag ctgtgggagg cacagtcacc |
| | | | 61 | atcagttgcc agtccagtga gagtgtttat aagaccaact acttatcctg gtttcagaag |
| | | | 121 | aaaccagggc agcctcccaa gctcctgatc tatgatgcat ccactctggc atctggggtc |
| | | | 181 | ccatcgcgct tcagcggcag tggatctggg acacagttca ctctcaccat cagcgacctg |
| | | | 241 | gagtgtgacg atgctgccac ttactactgt gcaggcggtt atagtgatga tattaatgct |
| | | | 301 | ttcggcggag ggaccgaggt ggtggtcaaa |
| 27 | 10-4 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 28 | 10-4 | VL | 1 | gatgtcgtga tgacccagac tccatccccc gtgtctgcac ctgtgggagg cacagtcacc |
| | | | 61 | atcaattgcc aggccagtca gaacatttac agcaatttag cctggtatca gcagaaacca |
| | | | 121 | gggcagcctc ccaagctcct gatctatggt gcatccactc tggcatctgg ggtctcatcg |
| | | | 181 | cggttcaaag gcagtagatc tgggacagag ttcactctca ccatcagcga cctggagtgt |
| | | | 241 | gccgatgctg ccacttatta ttgtcaaagc tatgtttata gtagtagtac tgctgatact |
| | | | 301 | ttcggcggag ggaccaaggt ggtcgtcgaa |
| 29 | 10-5 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 30 | 10-5 | VL | 1 | gatgtcgtga tgacccagac tccatccccc gtgtctgcac ctgtgggagg cacagtcacc |
| | | | 61 | atcaattgcc aggccagtca gaacatttac agcaatttag cctggtatca gcagaaacca |
| | | | 121 | gggcagcctc ccaagctcct gatctatggt gcatccactc tggcatctgg ggtctcatcg |
| | | | 181 | cggttcaaag gcagtagatc tgggacagag ttcactctca ccatcagcga cctggagtgt |
| | | | 241 | gccgatgctg ccacttatta ttgtcaaagc tatgtttata gtagtagtac tgctgatact |
| | | | 301 | ttcggcggag ggaccaaggt ggtcgtcgaa |
| 31 | 10-9 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 32 | 10-9 | VL | 1 | gcagccgtgc tgacccagac tccatcgtcc gtgtctgcag ctgtgggagg cacagtcacc |
| | | | 61 | atcagttgcc agtccagtga gagtgtttat aagaccaact acttatcctg gtttcagaag |
| | | | 121 | aaaccagggc agcctcccaa gctcctgatc tatgatgcat ccactctggc atctggggtc |
| | | | 181 | ccatcgcgct tcagcggcag tggatctggg acacagttca ctctcaccat cagcgacctg |
| | | | 241 | gagtgtgacg atgctgccac ttactactgt gcaggcggtt atagtgatga tattaatgct |
| | | | 301 | ttcggcggag ggaccgaggt ggaaatcaaa |
| 33 | 10-12 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 34 | 10-12 | VL | 1 | gagcttgtgc tgacccagac tgcatcgtcc gtgtctgcag ctgtgggagg cacagtcacc |
| | | | 61 | atcagttgcc agtccagtga gagtgtttat aagaccaact acttatcctg gtttcagaag |
| | | | 121 | aaaccagggc agcctcccaa gctcctgatc tatgatgcat ccactctggc atctggggtc |
| | | | 181 | ccatcgcgct tcagcggcag tggatctggg acacagttca ctctcaccat cagcgacctg |
| | | | 241 | gagtgtgacg atgctgccac ttactactgt gcaggcggtt atagtgatga tattaatgct |
| | | | 301 | ttcggcggag ggaccgaggt ggtggtcaaa |
| 35 | 10-12 | VL | 1 | gatgtcgtga tgacccagac tccatccccc gtgtctgcac ctgtgggagg cacagtcacc |
| | | | 61 | atcaattgcc aggccagtca gaacatttac agcaatttag cctggtatca gcagaaacca |
| | | | 121 | gggcagcctc ccaagctcct gatctatggt gcatccactc tggcatctgg ggtctcatcg |
| | | | 181 | cggttcaaag gcagtagatc tgggacagag ttcactctca ccatcagcga cctggagtgt |
| | | | 241 | gccgatgctg ccacttatta ttgtcaaagc tatgtttata gtagtagtac tgctgatact |
| | | | 301 | ttcggcggag ggaccaaggt ggtcgtcgaa |
| 36 | 12-1 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 37 | 12-1 | VL | 1 | gatgtcgtga tgacccagac tccatccccc gtgtctgcag ctgtgggagg cacagtcacc |
| | | | 61 | atcaattgcc aggccagtca gaacatttac agcaatttag cctggtatca gcagaaacca |
| | | | 121 | gggcagcctc ccaagctcct gatctatggt gcatccactc tggcatctgg ggtctcatcg |
| | | | 181 | cggttcaaag gcagtagatc tgggacagag ttcactctca ccatcagcga cctggagtgt |
| | | | 241 | gccgatgctg ccacttatta ttgtcaaagc tatgtttata gtagtagtac tgctgatact |
| | | | 301 | ttcggcggag ggaccaaggt ggtcgtcgaa |
| 38 | 12-3 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 39 | 12-3 | VL | 1 | gatgtcgtga tgacccagac tccatccccc gtgtctgcac ctgtgggagg cacagtcacc |
| | | | 61 | atcaattgcc aggccagtca gaacatttac agcaatttag cctggtatca gcagaaacca |
| | | | 121 | gggcagcctc ccaagctcct gatctatggt gcatccactc tggcatctgg ggtctcatcg |
| | | | 181 | cggttcaaag gcagtagatc tgggacagag ttcactctca ccatcagcga cctggagtgt |
| | | | 241 | gccgatgctg ccacttatta ttgtcaaagc tatgtttata gtagtagtac tgctgatact |
| | | | 301 | ttcggcggag ggaccaaggt ggtcgtcgaa |
| 40 | 12-8 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 41 | 12-8 | VL | 1 | gcagccgtgc tgacccagac tccatcgtcc gtgtctgcag ctgtgggagg cacagtcacc |
| | | | 61 | atcagttgcc agtccagtga gagtgtttat aagaccaact acttatcctg gtttcagaag |
| | | | 121 | aaaccagggc agcctcccaa gctcctgatc tatgatgcat ccactctggc atctggggtc |
| | | | 181 | ccatcgcgct tcagcggcag tggatctggg acacagttca ctctcaccat cagcgacctg |
| | | | 241 | gagtgtgacg atgctgccac ttactactgt gcaggcggtt atagtgatga tattaatgct |
| | | | 301 | ttcggcggag ggaccgaggt ggaaatcaaa |
| 42 | 12-10 | VH | 1 | cagtcgttgg aggagtccgg gggagacctg gtcaagcctg gggcatccct gacactcacc |
| | | | 61 | tgcacagcct ctggattctc cttcagtagc ggctactgga tatgctgggt ccgccaggct |
| | | | 121 | ccagggaagg ggctggagtg gatcgcgtgc atttatactg gtgatggtga cacttcctac |
| | | | 181 | gcgagctggg cgaaaggccg attcaccatc tccaaaacct cgtcgaccac ggtgactctg |
| | | | 241 | caaatgacca gtctgacagt cgcggacacg gccatttatt tttgtgcgag agatactagt |
| | | | 301 | ggtactttct attataattt gtggggccca ggcaccctgg tcgccgtctc ctca |
| 43 | 12-10 | VL | 1 | gcagccgtgc tgacccagac tccatcgtcc gtgtctgcag ctgtgggagg cacagtcacc |
| | | | 61 | atcagttgcc agtccagtga gagtgtttat aagaccaact acttatcctg gtttcagaag |
| | | | 121 | aaaccagggc agcctcccaa gctcctgatc tatgatgcat ccactctggc atctggggtc |
| | | | 181 | ccatcgcgct tcagcggcag tggatctggg acacagttca ctctcaccat cagcgacctg |
| | | | 241 | gagtgtgacg atgctgccac ttactactgt gcaggcggtt atagtgatga tattaatgct |
| | | | 301 | ttcggcggag ggaccgaggt ggaaatcaaa |
| 44 | 6.3 | VH | 1 | cagtcgttgg aggagtccgg gggaggcctg gtcaagcctg agggatccct gacactcacc |
| | | | 61 | tgcacagcct ctggattctc cttcggtagc ggctcctgga tatgttgggt ccgccaggct |
| | | | 121 | ccagggaagg ggctggagtg gatcgcatgc atttatggta gtggtagtgg tgacactgcc |
| | | | 181 | tacgcgacct gggcgaaagg ccgattcacc atctccaaaa cctcgtcgac cacggtgact |
| | | | 241 | ctgcaaatga ccagtctgac agccgcggac acggccacct atttctgtgc gagggacagt |
| | | | 301 | gattatgtgg actttgactt gtggggccca ggcaccctgg tcactgtctc ctca |
| 45 | 6.3 | VL | 1 | gcccaagtgc tgacccagac tccatcttcc acgtctgcgg ctgtgggagg cacagtcacc |
| | | | 61 | atcaactgcc agtccagtca gagtgtttat aagaacaact acttatcctg gtttcagcag |
| | | | 121 | aaaccagggc agcctcccaa gctcctgatc tatctggcat ccactctggc atctggggtc |
| | | | 181 | ccatcgcggt tcagcggcag tggatctggg acacagttca ctctcaccat cagcgacctg |
| | | | 241 | gagtgtgacg atgctgccac ttactactgt gcaggcggtt atagtgatga tatttgtact |
| | | | 301 | ttcggcggag ggaccgaggt ggtggtcaaa |

### Example 2. Screening Clones for Specific Binding to TAT Frataxin Fusion Molecule

The sequences of the heavy and light variable regions of the 10 high affinity monoclonal clones described in Example 1 were cloned into a different rabbit IgG backbone sequence to generate test plasmids. A sandwich ELISA was modified from use with a polyclonal anti-TAT capture to screen for clones with appropriate capture sensitivity for TAT containing proteins as follows: ELISA plates were coated with 504, 5 μg/ml of purified expressed antibodies from the clone cells transfected with test plasmids, and then blocked with a high protein buffer. 504, calibrators of buffer spiked with TAT-frataxin in concentrations ranging from 1000 ng/ml to 0.05 ng/ml were incubated in coated wells for 2 hrs at room temperature, and then washed. 504, of buffer containing a known Anti-Frataxin monoclonal antibody labelled with HRP was then incubated for 1 hr in the wells, followed by washing, and a 20 minute incubation with 1004, TMB substrate. The detection reaction was stopped with the addition of 1004, 1N H2SO4, and the plate was read at 450 nm. Curves were fitted using Softmax to determine sensitivity and compare antibodies expressed the clones.

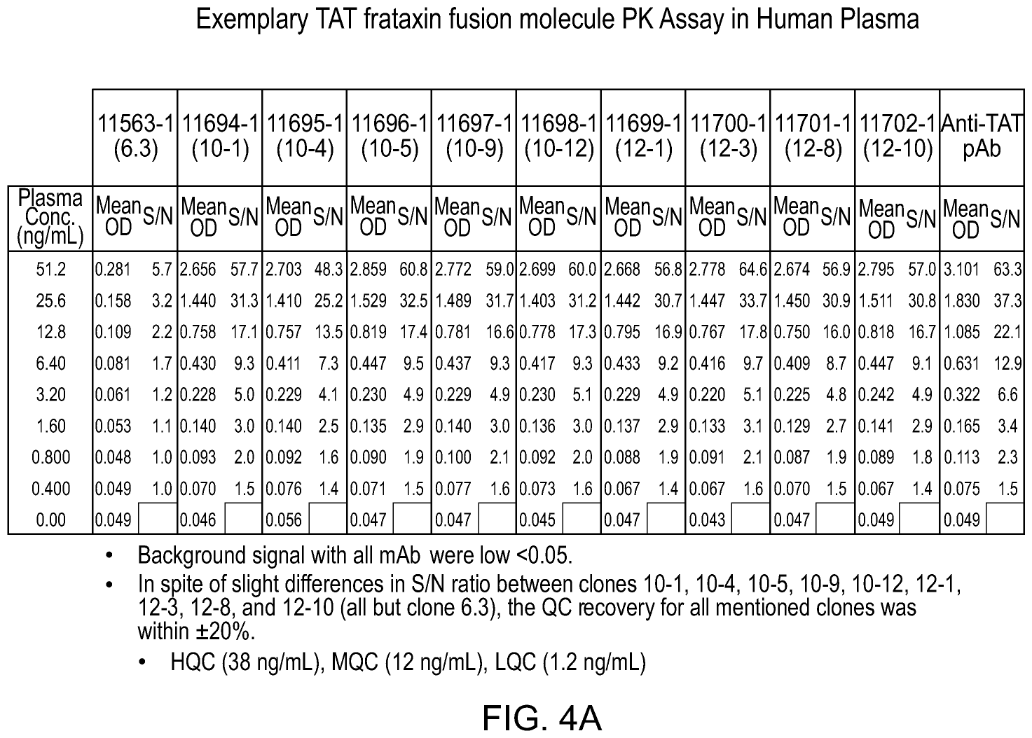

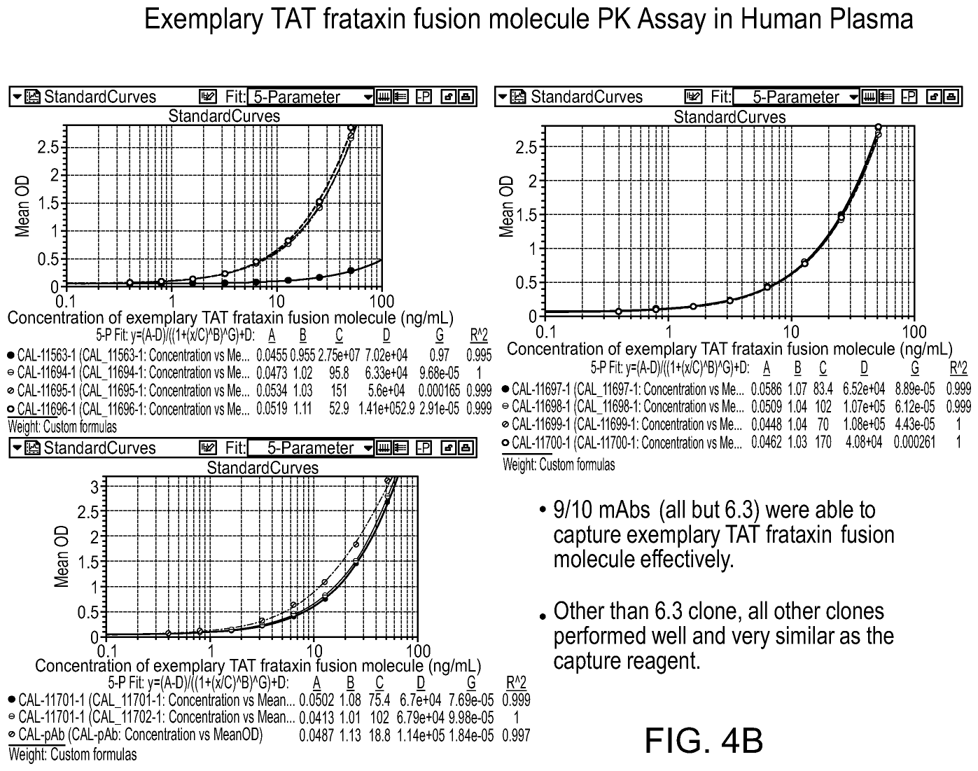

Nine out of 10 of the identified antibodies (all except antibody 6.3) were able to effectively capture an exemplary TAT fusion molecule comprising the TAT protein transduction domain and frataxin in a pharmacokinetic (PK) assay in human plasma (see FIG. 4A and FIG. 4B).

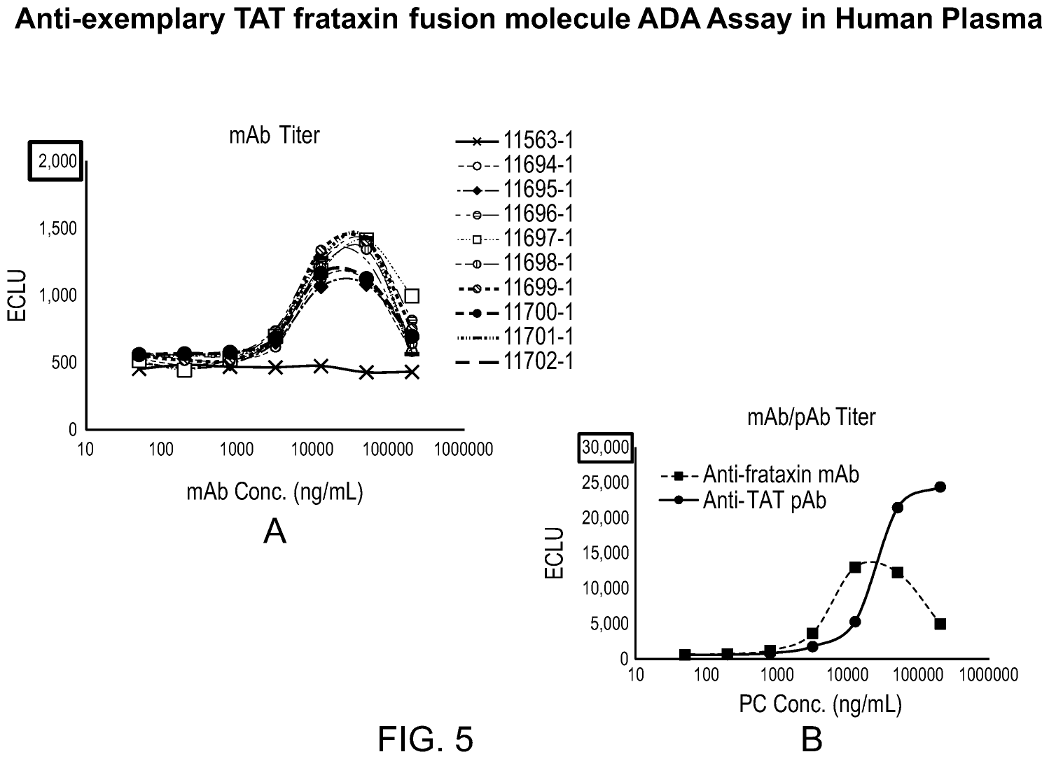

The antibodies were tested for specific binding to an exemplary TAT FXN fusion molecule in a binding assay in human plasma. The binding assay used was an ADA or bridging assay, generally as described in Example 3 below. The results of the binding assay are presented in FIG. 5A. The results demonstrate that, with the exception of one antibody (11563-1), all tested recombinant antibodies exhibited specific binding to the TAT FXN fusion molecule. The results also indicate that the sequences of the heavy and light chain variable regions of the anti-TAT monoclonal antibodies, when placed into different backbones, confer an ability to specifically bind TAT-FXN fusion molecule. This confirms the discovery of high affinity rabbit antibodies. All clones with the exception of clone 11563-1 show a hook effect at high antibody concentration, as expected with a bridging/sandwich assay where the capture is dependent on a single defined epitope (TAT).

### Example 3. Anti-TAT Polyclonal Antibody is Effective as a Positive Control in an ADA Assay

The goal of this experiment was to compare a rabbit polyclonal anti-TAT antibody generated by the methods described herein to a commercially available anti-frataxin antibody to determine whether the rabbit polyclonal anti-TAT antibody could be used in an Anti-Drug Antibody (ADA) assay to test for immunogenicity of an exemplary TAT frataxin fusion molecule.

In the assay, two labeled forms of an exemplary TAT frataxin fusion molecule (biotinylated and sulfotagged forms) were co-incubated with defined concentrations of antibody. The samples were incubated on a strepatividin plate, unbound molecules were washed away, and the plate was subsequently read to see the amount of labeled (sulfotagged) antibodies bound. This is a bridging strategy in which high affinity molecules which are able to bind two TAT frataxin molecules complete a “bridge” which results in signal being bound to the plate.

The results are presented in FIG. 5B. Specifically, the results demonstrate that the commercially available anti-frataxin monoclonal antibody showed increasing bridging signal up to around 10 μg/ml antibody input, and decreasing signal at higher concentrations, commonly described as a “hook effect”. The rabbott polyclonal anti-TAT antibody generated increasing signal up to ˜50 μg/ml and remained at maximal signal to a concentration of ˜200 m/ml. The results indicate that the polyclonal anti-TAT antibody can function well as a positive control in an ADA assay to screen, e.g., clinical samples for potential antibodies arising from treatment with a TAT frataxin fusion molecule.

Sequence Listing

| Sequence | |
|---|---|
| Identifier | Protein |
| SEQ ID NO: 1 | 10-1, 10-4, 10-5, 10-9, 12-1, 12-3, 12-8, 12-10 |
| | VH amino acid sequence |
| SEQ ID NO: 2 | 10-1, 10-4, 10-5, 10-9, 10-12, 12-1, 12-3, 12-8, |
| | 12-10 VH CDR1 amino acid sequence |
| SEQ ID NO: 3 | 10-1, 10-4, 10-5, 10-9, 10-12, 12-1, 12-3, 12-8, |
| | 12-10 VH CDR2 amino acid sequence |
| SEQ ID NO: 4 | 10-1, 10-4, 10-5, 10-9, 10-12, 12-1, 12-3, 12-8, |
| | 12-10 VH CDR3 amino acid sequence |
| SEQ ID NO: 5 | 10-1, 10-12 VL amino acid sequence |
| SEQ ID NO: 6 | 10-1, 10-9, 10-12, 12-8, 12-10 VL CDR1 amino |
| | acid sequence |
| SEQ ID NO: 7 | 10-1, 10-9, 10-12, 12-8, 12-10 VL CDR2 amino |
| | acid sequence |
| SEQ ID NO: 8 | 10-1, 10-9, 10-12, 12-8, 12-10 VL CDR3 |
| | amino acid sequence |
| SEQ ID NO: 9 | 10-4, 10-5, 12-1, 12-3 VL amino acid sequence |
| SEQ ID NO: 10 | 10-4, 10-5, 12-1, 12-3 VL CDR1 amino acid |
| | sequence |
| SEQ ID NO: 11 | 10-4, 10-5, 12-1, 12-3 VL CDR2 amino acid |
| | sequence |
| SEQ ID NO: 12 | 10-4, 10-5, 12-1, 12-3 VL CDR3 amino acid |
| | sequence |
| SEQ ID NO: 13 | 10-9, 12-8, 12-10 VL amino acid sequence |
| SEQ ID NO: 14 | 10-12 VH amino acid sequence |
| SEQ ID NO: 15 | 6.3 VH amino acid sequence |
| SEQ ID NO: 16 | 6.3 VH CDR1 amino acid sequence |
| SEQ ID NO: 17 | 6.3 VH CDR2 amino acid sequence |
| SEQ ID NO: 18 | 6.3 VH CDR3 amino acid sequence |
| SEQ ID NO: 19 | 6.3 VL amino acid sequence |
| SEQ ID NO: 20 | 6.3 VL CDR1 amino acid sequence |
| SEQ ID NO: 21 | 6.3 VL CDR2 amino acid sequence |
| SEQ ID NO: 22 | 6.3 VL CDR3 amino acid sequence |
| SEQ ID NO: 23 | TAT transduction domain amino acid sequence |
| SEQ ID NO: 24 | TAT protein (full length) |
| SEQ ID NO: 25 | 10-1 VH nucleic acid sequence |
| SEQ ID NO: 26 | 10-1 VL nucleic acid sequence |
| SEQ ID NO: 27 | 10-4 VH nucleic acid sequence |
| SEQ ID NO: 28 | 10-4 VL nucleic acid sequence |
| SEQ ID NO: 29 | 10-5 VH nucleic acid sequence |
| SEQ ID NO: 30 | 10-5 VL nucleic acid sequence |
| SEQ ID NO: 31 | 10-9 VH nucleic acid sequence |
| SEQ ID NO: 32 | 10-9 VL nucleic acid sequence |
| SEQ ID NO: 33 | 10-12 VH nucleic acid sequence |
| SEQ ID NO: 34 | 10-12 VL nucleic acid sequence_1 |
| SEQ ID NO: 35 | 10-12 VL nucleic acid sequence_2 |
| SEQ ID NO: 36 | 12-1 VH nucleic acid sequence |
| SEQ ID NO: 37 | 12-1 VL nucleic acid sequence |
| SEQ ID NO: 38 | 12-3 VH nucleic acid sequence |
| SEQ ID NO: 39 | 12-3 VL nucleic acid sequence |
| SEQ ID NO: 40 | 12-8 VH nucleic acid sequence |
| SEQ ID NO: 41 | 12-8 VL nucleic acid sequence |
| SEQ ID NO: 42 | 12-10 VH nucleic acid sequence |
| SEQ ID NO: 43 | 12-10 VL nucleic acid sequence |
| SEQ ID NO: 44 | 6.3 VH nucleic acid sequence |
| SEQ ID NO: 45 | 6.3 VL nucleic acid sequence |

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A binding protein comprising an antigen binding domain, said antigen binding domain comprising a heavy chain CDR3 domain comprising the amino acid sequence of SEQ ID NO: 4, wherein said binding protein is capable of binding a TAT protein transduction domain.

2. The binding protein of claim 1, wherein said antigen binding domain further comprises a heavy chain CDR2 domain comprising the amino acid sequence of SEQ ID NO: 3.

3. The binding protein of claim 1 or 2, wherein said antigen binding domain further comprises a heavy chain CDR1 domain comprising the amino acid sequence of SEQ ID NO: 2.

4. The binding protein of any one of claims 1-3, wherein said antigen binding domain further comprises a light chain CDR3 domain comprising the amino acid sequence selected from the group consisting of SEQ ID NO: 8 and SEQ ID NO: 12.

5. The binding protein of any one of claims 1-4, wherein said antigen binding domain further comprises a light chain CDR2 domain comprising the amino acid sequence selected from the group consisting of SEQ ID NO: 7 and SEQ ID NO:11.

6. The binding protein of any one of claims 1-5, wherein said antigen binding domain further comprises a light chain CDR1 domain comprising the amino acid sequence selected from the group consisting of SEQ ID NO: 6 and SEQ ID NO: 10.

7. A binding protein comprising an antigen binding domain, said antigen binding domain comprising:
a heavy chain variable region comprising a CDR3 domain comprising the amino acid sequence set forth in SEQ ID NO: 4, a CDR2 domain comprising the amino acid sequence set forth in SEQ ID NO: 3, and a CDR1 domain comprising the amino acid sequence set forth in SEQ ID NO: 2; and
a light chain variable region comprising a CDR3 domain comprising the amino acid sequence set forth in SEQ ID NO: 8, a CDR2 domain comprising the amino acid sequence set forth in SEQ ID NO: 7, and a CDR1 domain comprising the amino acid sequence set forth in SEQ ID NO: 6; or a light chain variable region comprising a CDR3 domain comprising the amino acid sequence set forth in SEQ ID NO: 12, a CDR2 domain comprising the amino acid sequence set forth in SEQ ID NO: 11, and a CDR1 domain comprising the amino acid sequence set forth in SEQ ID NO: 10,
wherein said binding protein is capable of binding a TAT protein transduction domain.

8. The binding protein of claim any one of claims 1-7, wherein said antigen binding domain comprises a heavy chain variable region comprising the amino acid sequence set forth in SEQ ID NO: 1 or SEQ ID NO: 14.

9. The binding protein of any one of claims 1-8, wherein said antigen binding domain comprises a light chain variable region comprising the amino acid sequence set forth in SEQ ID NO: 5, a light chain variable region comprising the amino acid sequence set forth in SEQ ID NO: 9, or a light chain variable region comprising the amino acid sequence set forth in SEQ ID NO: 13.

10. The binding protein of any one of claims 1-9, wherein said antigen binding domain comprises a heavy chain variable region comprising the amino acid sequence set forth in SEQ ID NO: 1 and a light chain variable region comprising the amino acid sequence set forth in SEQ ID NO: 5.

11. The binding protein of any one of claims 1-9, wherein said antigen binding domain comprises a heavy chain variable region comprising the amino acid sequence set forth in SEQ ID NO: 1 and a light chain variable region comprising the amino acid sequence set forth in SEQ ID NO: 9.

12. The binding protein of any one of claims 1-9, wherein said antigen binding domain comprises a heavy chain variable region comprising the amino acid sequence set forth in SEQ ID NO: 1 and a light chain variable region comprising the amino acid sequence set forth in SEQ ID NO: 13.

13. The binding protein of any one of claims 1-9, wherein said antigen binding domain comprises a heavy chain variable region comprising the amino acid sequence set forth in SEQ ID NO: 14 and a light chain variable region comprising the amino acid sequence set forth in SEQ ID NO: 5.

14. A binding protein comprising an antigen binding domain, said antigen binding domain comprising a heavy chain CDR3 domain comprising the amino acid sequence of SEQ ID NO: 18, wherein said binding protein is capable of binding a TAT protein transduction domain.

15. The binding protein of claim 14, wherein said antigen binding domain further comprises a heavy chain CDR2 domain comprising the amino acid sequence of SEQ ID NO: 17.

16. The binding protein of claim 14 or 15, wherein said antigen binding domain further comprises a heavy chain CDR1 domain comprising the amino acid sequence of SEQ ID NO: 16.

17. The binding protein of any one of claims 14-16, wherein said antigen binding domain further comprises a light chain CDR3 domain comprising the amino acid sequence of SEQ ID NO: 22.

18. The binding protein of any one of claims 14-17, wherein said antigen binding domain further comprises a light chain CDR2 domain comprising the amino acid sequence of SEQ ID NO: 21.

19. The binding protein of any one of claims 14-18, wherein said antigen binding domain further comprises a light chain CDR1 domain comprising the amino acid sequence of SEQ ID NO: 20.

20. A binding protein comprising an antigen binding domain, said antigen binding domain comprising:
a heavy chain variable region comprising a CDR3 domain comprising the amino acid sequence set forth in SEQ ID NO: 18, a CDR2 domain comprising the amino acid sequence set forth in SEQ ID NO: 17, and a CDR1 domain comprising the amino acid sequence set forth in SEQ ID NO: 16; and
a light chain variable region comprising a CDR3 domain comprising the amino acid sequence set forth in SEQ ID NO: 22, a CDR2 domain comprising the amino acid sequence set forth in SEQ ID NO: 21, and a CDR1 domain comprising the amino acid sequence set forth in SEQ ID NO: 20,
wherein said binding protein is capable of binding a TAT protein transduction domain.

21. The binding protein of claim any one of claims 14-20, wherein said antigen binding domain comprises a heavy chain variable region comprising the amino acid sequence set forth in SEQ ID NO: 15.

22. The binding protein of any one of claims 14-21, wherein said antigen binding domain comprises a light chain variable region comprising the amino acid sequence set forth in SEQ ID NO: 19.

23. The binding protein of any one of claims 14-22, wherein said antigen binding domain comprises a heavy chain variable region comprising the amino acid sequence set forth in SEQ ID NO: 15 and a light chain variable region comprising the amino acid sequence set forth in SEQ ID NO: 19.

24. The binding protein of any one of claims 1-23, wherein the TAT protein transduction domain comprises the amino acid sequence of SEQ ID NO: 23.

25. The binding protein of any one of claims 1-23, wherein the TAT protein transduction domain consists essentially of the amino acid sequence of SEQ ID NO: 23.

26. The binding protein of any one of claims 1-25, wherein the TAT protein transduction domain is covalently linked to a cargo moiety.

27. The binding protein of claim 26, wherein the cargo moiety is a polypeptide.

28. The binding protein of claim 26, wherein the cargo moiety is a frataxin polypeptide.

29. The binding protein of claim 26, wherein the cargo moiety is an antibody.

30. The binding protein of claim 26, wherein the cargo moiety is a nucleic acid.

31. The binding protein of claim 30, wherein the nucleic acid is an siRNA, shRNA, miRNA, phosphorothioate modified RNA, aptamer, phosphorodiamidate morpholino oligomer (PMO), or any combination thereof.

32. The binding protein of claim 26, wherein the cargo moiety is a small molecule, a liposome enclosing protein, a radionuclide or radionuclide labeled compound, or any combination thereof.

33. A binding protein comprising an antigen binding domain, wherein said binding protein is capable of binding to a TAT protein transduction domain that is covalently linked to a cargo moiety.

34. The binding protein of claim 33, wherein said antigen binding domain binds to an epitope comprising the amino acid residues of SEQ ID NO: 23.

35. The binding protein of claim 33, wherein the cargo moiety is a polypeptide.

36. The binding protein of claim 33, wherein the cargo moiety is a frataxin polypeptide.

37. The binding protein of claim 33, wherein the cargo moiety is an antibody.

38. The binding protein of claim 33, wherein the cargo moiety is a nucleic acid.

39. The binding protein of claim 38, wherein the nucleic acid is an siRNA, shRNA, miRNA, phosphorothioate modified RNA, aptamer, phosphorodiamidate morpholino oligomer (PMO), or any combination thereof.

40. The binding protein of claim 33, wherein the cargo moiety is a small molecule, a liposome enclosing protein, a radionuclide or radionuclide labeled compound, or any combination thereof.

41. The binding protein of any one of claims 1-40, wherein said binding protein is an antibody.

42. An antibody construct comprising the binding protein of any one of claims 1-41, said antibody construct further comprising a linker polypeptide or an immunoglobulin constant domain.

43. The antibody construct according to claim 42, wherein said binding protein is selected from the group consisting of: an immunoglobulin molecule, a monoclonal antibody, a murine antibody, a chimeric antibody, a CDR-grafted antibody, a humanized antibody, a single domain antibody, a Fv, a disulfide linked Fv, a scFv, a diabody, a Fab, a Fab′, a F(ab′)₂, a multispecific antibody, a dual specific antibody, and a bispecific antibody.

44. The antibody construct according to any one of claims 42 and 43, wherein said binding protein comprises a heavy chain immunoglobulin constant domain selected from the group consisting of: a IgM constant domain, a IgG₄ constant domain, a IgG₁ constant domain, a IgE constant domain, a IgG₂ constant domain, a IgG₃ constant domain and a IgA constant domain.

45. The antibody construct according to claim 44, wherein said binding protein comprises a IgG₁ constant domain.

46. The antibody construct according to claim 44, wherein said heavy chain immunoglobulin constant domain is not IgM.

47. An isolated nucleic acid encoding a binding protein amino acid sequence of any one of claims 1-41.

48. An isolated nucleic acid encoding an antibody construct amino acid sequence of any one of claims 42-46.

49. A vector comprising an isolated nucleic acid according to claim 47 or 48.

50. The vector of claim 49 wherein said vector is selected from the group consisting of pcDNA, pTT, pTT3, pEFBOS, pBV, pJV, pBJ, pGEX, VSV, pBR322, pCMV-HA, pEN, YAC, BAC, Bacteriophage Lamda, Phagemid, pCAS9, pCEN6, pYES1L, p3HPRT1, pFN2A, pBC, pTZ, pGEM, pGEMK, pEX, pSAR, pCEP, Cosmids, pBluescript, pKJK, pFloxin, pCP, pHR, pUC, and pMAL.

51. A host cell comprising a vector according to claim 49 or 50.

52. The host cell according to claim 51, wherein said host cell is a prokaryotic cell or a eukaryotic cell.

53. The host cell according to claim 51, wherein said prokaryotic host cell is E. Coli.

54. The host cell according to claim 51, wherein said eukaryotic cell is selected from the group consisting of a protist cell, an insect cell, an animal cell, a plant cell and a fungal cell.

55. The host cell according to claim 51, wherein said animal cell is a mammalian cell or an avian cell.

56. The host cell according to claim 51, wherein said host cell is selected from the group consisting of a CHO cell, a COS cell, a yeast cell, an insect Sf9 cell, HEK-293 cell, ExpiCHO cell, Expi-293f cell, and E. coli cell.

57. The host cell according to claim 56, wherein said yeast cell is Saccharomyces cerevisiae.

58. A method of producing an antibody, or antigen binding portion thereof, comprising culturing the host cell of any one of claims 51-57 in culture medium so that said isolated nucleic acid is expressed and said antibody is produced.

59. An antibody produced according to the method of claim 58.

60. A transgenic mouse comprising the host cell of any one of claims 51-57, wherein the mouse expresses a polypeptide encoded by the nucleic acid, or an antigen binding portion thereof, that binds to a TAT protein transduction domain.

61. A hybridoma that produces the antibody construct of any one of claims 42-46.

62. The binding protein of any one of claims 1-41, which is immobilized on a solid support.

63. The binding protein of claim 62, wherein the solid support is a plate, a bead, or a chromatography resin.

64. The binding protein of claim 63, wherein the bead or chromatography resin comprises protein A agarose or protein G agarose.

65. The binding protein of any one of claims 1-41, which is conjugated to a detection molecule.

66. The binding protein of claim 65, wherein the detection molecule is horseradish peroxidase, sulfotag, alkaline phosphatase, cresol violet, quantum dots, FITC, an infrared molecule, a radiolabel, or an EPR spin tracer label.

67. A method for detecting and/or quantifying the level of a TAT fusion molecule in a sample, comprising contacting the sample with a binding protein of any one of claims 1-41 under conditions such that the binding protein binds to TAT protein transduction domain in the sample, to thereby detect and/or quantify the level of the TAT fusion molecule in the sample.

68. The method of claim 67, wherein the sample is a biological sample.

69. The method of claim 68, wherein the biological sample is a liquid sample or a tissue sample.

70. The method of claim 68, wherein the TAT fusion molecule comprises a TAT protein transduction domain covalently linked to a cargo moiety.

71. The method of claim 70, wherein the cargo moiety is a polypeptide.

72. The method of claim 70, wherein the cargo moiety is a frataxin polypeptide.

73. The method of claim 70, wherein the cargo moiety is an antibody.

74. The method of claim 70, wherein the cargo moiety is a nucleic acid.

75. The binding protein of claim 74, wherein the nucleic acid is an siRNA, shRNA, miRNA, phosphorothioate modified RNA, aptamer, phosphorodiamidate morpholino oligomer (PMO), or any combination thereof.

76. The binding protein of claim 70, wherein the cargo moiety is a small molecule, a liposome enclosing protein, a radionuclide or radionuclide labeled compound, or any combination thereof.

77. The method of claim 67, further comprising assessing the stability of the TAT fusion molecule.

78. A method of isolating and/or purifying a TAT fusion molecule present in a mixture, wherein said TAT fusion molecule comprises a TAT protein transduction domain covalently linked to a cargo moiety, comprising (a) contacting said mixture comprising the TAT fusion molecule with the immobilized binding protein of any one of claims 62-64 under conditions such that the TAT fusion molecule binds to the immobilized binding protein; (b) eluting said TAT fusion molecule from the immobilized binding protein.

79. A kit for comprising at least one reagent specific for the detection of a level of a TAT protein transduction domain, wherein the detection reagent is a binding protein of any one of claims 1-41.

80. The kit of claim 79, wherein the TAT protein transduction domain is covalently linked to a cargo moiety.

81. The kit of claim 79 or 80, wherein the kit further comprises instructions for the detection, quantitation or characterization of the TAT protein transduction domain.

82. A method of inhibiting the translocation of a TAT fusion molecule across a cell membrane, comprising contacting the TAT fusion molecule with an antigen binding protein of any one of claims 1-41, thereby inhibiting translocation of the TAT fusion molecule across the cell membrane.

83. A method of inhibiting the activity of HIV-TAT protein in a subject, comprising administering to the subject an antigen binding protein of any one of claims 1-41, thereby inhibiting activity of the HIV-TAT protein in the subject.


SEQUENCE LISTING (47 sequences)

SEQ ID NO: 1 - protein, 118 aa, Artificial Sequence
  source: /note=“Description of Artificial Sequence Synthetic polypeptide”
QSLEESGGDLVKPGASLTLTCTASGFSFSSGYWICWVRQAPGKGLEWIACIYTGDGDTSYASWAKGRFTISKTSSTTVTLQMTSLTVADTAIYFCARDTSGTFYYNLWGPGTLVAVSS

SEQ ID NO: 2 - protein, 8 aa, Artificial Sequence
  source: /note=“Description of Artificial Sequence Synthetic peptide”
FSSGYWIC

SEQ ID NO: 3 - protein, 17 aa, Artificial Sequence
  source: /note=“Description of Artificial Sequence Synthetic peptide”
CIYTGDGDTSYASWAKG

SEQ ID NO: 4 - protein, 10 aa, Artificial Sequence
  source: /note=“Description of Artificial Sequence Synthetic peptide”
DTSGTFYYNL

SEQ ID NO: 5 - protein, 110 aa, Artificial Sequence
  source: /note=“Description of Artificial Sequence Synthetic polypeptide”
ELVLTQTASSVSAAVGGTVTISCQSSESVYKTNYLSWFQKKPGQPPKLLIYDASTLASGVPSRFSGSGSGTQFTLTISDLECDDAATYYCAGGYSDDINAFGGGTEVVVK

SEQ ID NO: 6 - protein, 13 aa, Artificial Sequence
  source: /note=“Description of Artificial Sequence Synthetic peptide”
QSSESVYKTNYLS

SEQ ID NO: 7 - protein, 7 aa, Artificial Sequence
  source: /note=“Description of Artificial Sequence Synthetic peptide”
DASTLAS

SEQ ID NO: 8 - protein, 10 aa, Artificial Sequence
  source: /note=“Description of Artificial Sequence Synthetic peptide”
AGGYSDDINA

SEQ ID NO: 9 - protein, 110 aa, Artificial Sequence
  source: /note=“Description of Artificial Sequence Synthetic polypeptide”
DVVMTQTPSPVSAPVGGTVTINCQASQNIYSNLAWYQQKPGQPPKLLIYGASTLASGVSSRFKGSRSGTEFTLTISDLECADAATYYCQSYVYSSSTADTFGGGTKVVVE

SEQ ID NO: 10 - protein, 11 aa, Artificial Sequence
  source: /note=“Description of Artificial Sequence Synthetic peptide”
QASQNIYSNLA

SEQ ID NO: 11 - protein, 7 aa, Artificial Sequence
  source: /note=“Description of Artificial Sequence Synthetic peptide”
GASTLAS

SEQ ID NO: 12 - protein, 12 aa, Artificial Sequence
  source: /note=“Description of Artificial Sequence Synthetic peptide”
QSYVYSSSTADT

SEQ ID NO: 13 - protein, 110 aa, Artificial Sequence
  source: /note=“Description of Artificial Sequence Synthetic polypeptide”
AAVLTQTPSSVSAAVGGTVTISCQSSESVYKTNYLSWFQKKPGQPPKLLIYDASTLASGVPSRFSGSGSGTQFTLTISDLECDDAATYYCAGGYSDDINAFGGGTEVEIK

SEQ ID NO: 14 - protein, 118 aa, Artificial Sequence; identical to SEQ ID NO: 1
  source: /note=“Description of Artificial Sequence Synthetic polypeptide”

SEQ ID NO: 15 - protein, 118 aa, Artificial Sequence
  source: /note=“Description of Artificial Sequence Synthetic polypeptide”
QSLEESGGGLVKPEGSLTLTCTASGFSFGSGSWICWVRQAPGKGLEWIACIYGSGSGDTAYATWAKGRFTISKTSSTTVTLQMTSLTAADTATYFCARDSDYVDFDLWGPGTLVTVSS

SEQ ID NO: 16 - protein, 8 aa, Artificial Sequence
  source: /note=“Description of Artificial Sequence Synthetic peptide”
FGSGSWIC

SEQ ID NO: 17 - protein, 18 aa, Artificial Sequence
  source: /note=“Description of Artificial Sequence Synthetic peptide”
CIYGSGSGDTAYATWAKG

SEQ ID NO: 18 - protein, 9 aa, Artificial Sequence
  source: /note=“Description of Artificial Sequence Synthetic peptide”
DSDYVDFDL

SEQ ID NO: 19 - protein, 110 aa, Artificial Sequence
  source: /note=“Description of Artificial Sequence Synthetic polypeptide”
AQVLTQTPSSTSAAVGGTVTINCQSSQSVYKNNYLSWFQQKPGQPPKLLIYLASTLASGVPSRFSGSGSGTQFTLTISDLECDDAATYYCAGGYSDDICTFGGGTEVVVK

SEQ ID NO: 20 - protein, 13 aa, Artificial Sequence
  source: /note=“Description of Artificial Sequence Synthetic peptide”
QSSQSVYKNNYLS

SEQ ID NO: 21 - protein, 7 aa, Artificial Sequence
  source: /note=“Description of Artificial Sequence Synthetic peptide”
LASTLAS

SEQ ID NO: 22 - protein, 10 aa, Artificial Sequence
  source: /note=“Description of Artificial Sequence Synthetic peptide”
AGGYSDDICT

SEQ ID NO: 23 - protein, 11 aa, Human immunodeficiency virus 1
YGRKKRRQRRR

SEQ ID NO: 24 - protein, 86 aa, Human immunodeficiency virus 1
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 25 - DNA, 354 bp, Artificial Sequence
  source: /note=“Description of Artificial Sequence Synthetic polynucleotide”
cagtcgttggaggagtccgggggagacctggtcaagcctggggcatccctgacactcacctgcacagcctctggattctccttcagtagcggctactggatatgctgggtccgccaggctccagggaaggggctggagtggatcgcgtgcatttatactggtgatggtgacacttcctacgcgagctgggcgaaaggccgattcaccatctccaaaacctcgtcgaccacggtgactctgcaaatgaccagtctgacagtcgcggacacggccatttatttttgtgcgagagatactagtggtactttctattataatttgtggggcccaggcaccctggtcgccgtctcctca

SEQ ID NO: 26 - DNA, 330 bp, Artificial Sequence
  source: /note=“Description of Artificial Sequence Synthetic polynucleotide”
gagcttgtgctgacccagactgcatcgtccgtgtctgcagctgtgggaggcacagtcaccatcagttgccagtccagtgagagtgtttataagaccaactacttatcctggtttcagaagaaaccagggcagcctcccaagctcctgatctatgatgcatccactctggcatctggggtcccatcgcgcttcagcggcagtggatctgggacacagttcactctcaccatcagcgacctggagtgtgacgatgctgccacttactactgtgcaggcggttatagtgatgatattaatgctttcggcggagggaccgaggtggtggtcaaa

SEQ ID NO: 27 - DNA, 354 bp, Artificial Sequence; identical to SEQ ID NO: 25
  source: /note=“Description of Artificial Sequence Synthetic polynucleotide”

SEQ ID NO: 28 - DNA, 330 bp, Artificial Sequence
  source: /note=“Description of Artificial Sequence Synthetic polynucleotide”
gatgtcgtgatgacccagactccatcccccgtgtctgcacctgtgggaggcacagtcaccatcaattgccaggccagtcagaacatttacagcaatttagcctggtatcagcagaaaccagggcagcctcccaagctcctgatctatggtgcatccactctggcatctggggtctcatcgcggttcaaaggcagtagatctgggacagagttcactctcaccatcagcgacctggagtgtgccgatgctgccacttattattgtcaaagctatgtttatagtagtagtactgctgatactttcggcggagggaccaaggtggtcgtcgaa

SEQ ID NO: 29 - DNA, 354 bp, Artificial Sequence; identical to SEQ ID NO: 25
  source: /note=“Description of Artificial Sequence Synthetic polynucleotide”

SEQ ID NO: 30 - DNA, 330 bp, Artificial Sequence; identical to SEQ ID NO: 28
  source: /note=“Description of Artificial Sequence Synthetic polynucleotide”

SEQ ID NO: 31 - DNA, 354 bp, Artificial Sequence; identical to SEQ ID NO: 25
  source: /note=“Description of Artificial Sequence Synthetic polynucleotide”

SEQ ID NO: 32 - DNA, 330 bp, Artificial Sequence
  source: /note=“Description of Artificial Sequence Synthetic polynucleotide”
gcagccgtgctgacccagactccatcgtccgtgtctgcagctgtgggaggcacagtcaccatcagttgccagtccagtgagagtgtttataagaccaactacttatcctggtttcagaagaaaccagggcagcctcccaagctcctgatctatgatgcatccactctggcatctggggtcccatcgcgcttcagcggcagtggatctgggacacagttcactctcaccatcagcgacctggagtgtgacgatgctgccacttactactgtgcaggcggttatagtgatgatattaatgctttcggcggagggaccgaggtggaaatcaaa

SEQ ID NO: 33 - DNA, 354 bp, Artificial Sequence; identical to SEQ ID NO: 25
  source: /note=“Description of Artificial Sequence Synthetic polynucleotide”

SEQ ID NO: 34 - DNA, 330 bp, Artificial Sequence; identical to SEQ ID NO: 26
  source: /note=“Description of Artificial Sequence Synthetic polynucleotide”

SEQ ID NO: 35 - DNA, 330 bp, Artificial Sequence; identical to SEQ ID NO: 28
  source: /note=“Description of Artificial Sequence Synthetic polynucleotide”

SEQ ID NO: 36 - DNA, 354 bp, Artificial Sequence; identical to SEQ ID NO: 25
  source: /note=“Description of Artificial Sequence Synthetic polynucleotide”

SEQ ID NO: 37 - DNA, 330 bp, Artificial Sequence; identical to SEQ ID NO: 28
  source: /note=“Description of Artificial Sequence Synthetic polynucleotide”

SEQ ID NO: 38 - DNA, 354 bp, Artificial Sequence; identical to SEQ ID NO: 25
  source: /note=“Description of Artificial Sequence Synthetic polynucleotide”

SEQ ID NO: 39 - DNA, 330 bp, Artificial Sequence; identical to SEQ ID NO: 28
  source: /note=“Description of Artificial Sequence Synthetic polynucleotide”

SEQ ID NO: 40 - DNA, 354 bp, Artificial Sequence; identical to SEQ ID NO: 25
  source: /note=“Description of Artificial Sequence Synthetic polynucleotide”

SEQ ID NO: 41 - DNA, 330 bp, Artificial Sequence; identical to SEQ ID NO: 32
  source: /note=“Description of Artificial Sequence Synthetic polynucleotide”

SEQ ID NO: 42 - DNA, 354 bp, Artificial Sequence; identical to SEQ ID NO: 25
  source: /note=“Description of Artificial Sequence Synthetic polynucleotide”

SEQ ID NO: 43 - DNA, 330 bp, Artificial Sequence; identical to SEQ ID NO: 32
  source: /note=“Description of Artificial Sequence Synthetic polynucleotide”

SEQ ID NO: 44 - DNA, 354 bp, Artificial Sequence
  source: /note=“Description of Artificial Sequence Synthetic polynucleotide”
cagtcgttggaggagtccgggggaggcctggtcaagcctgagggatccctgacactcacctgcacagcctctggattctccttcggtagcggctcctggatatgttgggtccgccaggctccagggaaggggctggagtggatcgcatgcatttatggtagtggtagtggtgacactgcctacgcgacctgggcgaaaggccgattcaccatctccaaaacctcgtcgaccacggtgactctgcaaatgaccagtctgacagccgcggacacggccacctatttctgtgcgagggacagtgattatgtggactttgacttgtggggcccaggcaccctggtcactgtctcctca

SEQ ID NO: 45 - DNA, 330 bp, Artificial Sequence
  source: /note=“Description of Artificial Sequence Synthetic polynucleotide”
gcccaagtgctgacccagactccatcttccacgtctgcggctgtgggaggcacagtcaccatcaactgccagtccagtcagagtgtttataagaacaactacttatcctggtttcagcagaaaccagggcagcctcccaagctcctgatctatctggcatccactctggcatctggggtcccatcgcggttcagcggcagtggatctgggacacagttcactctcaccatcagcgacctggagtgtgacgatgctgccacttactactgtgcaggcggttatagtgatgatatttgtactttcggcggagggaccgaggtggtggtcaaa

SEQ ID NO: 46 - protein, 13 aa, Artificial Sequence
  source: /note=“Description of Artificial Sequence Synthetic peptide”
MYGRKKRRQRRRC

SEQ ID NO: 47 - protein, 9 aa, Artificial Sequence
  source: /note=“Description of Artificial Sequence Synthetic peptide”
GLNQIWNVK